(19) United States
(12) Patent Application Publication
(10) Pub. No.: US 2026/0263533 A1
(43) Pub. Date: Sep. 10, 2026

(54) COMBINATION OF BIFIDOBACTERIUM AND FUCOSYLATED HMO FOR USE IN INCREASING NMN OR NAD+

(71) Applicants: DSM IP Assets B.V., Maastricht (NL); DANSTAR FERMENT AG, Zug (CH)

(72) Inventors: Leigh MATANO, Hørsholm (DK); Danica BAJIC, Hørsholm (DK); Ghislain SCHYNS, Hørsholm (DK); Sylvie BINDA, Blagnac (FR)

(21) Appl. No.: 18/994,229

(22) PCT Filed: Jul. 14, 2023
(86) PCT No.: PCT/EP2023/069717

§ 371 Date: Jan. 14, 2025

Related U.S. Application Data
(60) Provisional application No. 63/389,778, filed on Jul. 15, 2022.
(60) Provisional application No. 63/386,729, filed on Dec. 9, 2022.

(51) Int. Cl. A61K 35/74; A61K 8/73; A61K 8/99; A61K 31/715; A61P 39/06; A61Q 19/00

(52) U.S. Cl. CPC A61K 35/74; A61K 8/73; A61K 8/99; A61K 31/715; A61P 39/06; A61Q 19/00

(57) ABSTRACT

The present disclosure relates to a method of increasing nicotinamide mononucleotide (NMN) and/or nicotinamide adenine dinucleotide (NAD+) in the gut of a subject by administering a combination comprising at least one Bifidobacterium sp. capable of growing on a sialylated HMO and having a functional de novo nicotinamide adenine dinucleotide (nad) operon (nadC, nadD and nadE) and at least one fucosylated oligosaccharide to said subject.

### FIELD

The present disclosure relates to a method of increasing nicotinamide mononucleotide (NMN) and/or nicotinamide adenine dinucleotide (NAD+) in the gut of a subject by administering a combination comprising at least one Bifidobacterium sp. capable of growing on a sialylated HMO and having a functional de novo nicotinamide adenine dinucleotide (nad) operon (nadC, nadD and nadE) and at least one fucosylated oligosaccharide to said subject. Increasing NMN and/or NAD+ in the gut can increase the systemic NAD+ pool in a subject and be useful for treating or preventing a mitochondria-related disease, neurodegenerative diseases, bacterial infections, reducing inflammation symptoms and in improving muscle regeneration. The disclosure also relates to a composition of Bifidobacterium sp. capable of growing on a sialylated HMO and having a functional de novo nicotinamide adenine dinucleotide (nad) pathway and at least one fucosylated oligosaccharide.

### DEPOSITS

Bifidobacterium bifidum DSM 32403 (Biocare Copenhagen, a fully owned DSM affiliate) is a preferred strain according to the present disclosure. It has been deposited at Leibniz Institute DSMZ-German Collection of Microorganisms and Cell Cultures, Inhoffenstr. 7B, D-38124 Braunschweig, Germany, according to the Budapest Treaty on 15 Dec. 2016 and accepted by the Depository authority 3. January 2017. The accession number given by the International Depository Authority is DSM 32403. The strain has been isolated from a Human in the United States.

Bifidobacterium bifidum HA-132 (Lallemand Health Solutions Inc.) is another preferred strain according to the present disclosure. It has been deposited at Collection Nationale de Cultures de Microorganismes (CNCM; 25 Rue du Docteur Roux F-75724 Paris Cedex 15, France), according to the Budapest Treaty on Sep. 19, 2022, under the accession number CNCM I-5898.

### BACKGROUND

The human gut microbiome is composed of bacteria, archaea, viruses, and eukaryotic microbes that reside inter alia in the gut. These microbes have tremendous potential to impact our physiology, both in health and in disease. The microbiota of the human intestine is a complex and very dynamic microbial ecosystem, which is considered to serve numerous important functions for its human host, including protection against pathogens, induction of immune regulatory functions, nutrient processing and metabolic functions, these basic functions, affect directly or indirectly most of our physiologic functions. Various gut bacteria, including certain Bifidobacterium spp., can feed on oligosaccharides such as fructo-oligosaccharides (FOS), galacto-oligosaccharides (GOS) and human milk oligosaccharides (HMOs), whereas these structures are indigestible by humans. Due to the ability to feed beneficial microorganisms and induce the growth or activity of these, certain oligosaccharides are referred to as prebiotics.

Human milk oligosaccharides (HMOs) are a heterogeneous mixture of soluble glycans found in human milk. They are the third most abundant solid component after lactose and lipids in human milk and are present in concentrations of 5-25 g/l. Certain HMOs are believed to be important for the development of the infant gut microbiota in particular by increasing the predominance of bifidobacteria. This is viewed as beneficial for infants because some strains of Bifidobacterium species may have a positive effect on gut health.

More recently, the potential benefits of HMOs in adult health are receiving increased attention (see for example WO2018/157900 and WO2018/207110), both in terms of increasing the prevalence of beneficial bacteria in the gut, which in turn produce metabolites such as short chain fatty acids (SCFAs), of which for example butyrate, propionate, and acetate play significant roles both in gut and metabolic health, but also as signaling molecules in the brain-gut axis.

Some species of bifidobacteria may be among the most beneficial probiotics. As an example, certain strains of B. bifidum and B. longum may have immunomodulatory properties and may provide a protective effect for suppressing certain pathogens.

The benefits of providing certain combinations of prebiotics and probiotics, that may potentially be considered to be synbiotics or synbiotic compositions, may provide health benefits.

Various combinations of Bifidobacterium spp. with specific HMOs have often been described primarily in the context of supplements for infant formula or for promoting certain bifidobacteria in infants.

WO2021/260162 describes a combination of Bifidobacterium longum subsp. longum and Lacto-N-tetraose (LNT) for promoting the growth of beneficial bacteria in the gut of infants (<12 months) or young children (between one and less than three years).

WO2021/260163 describes a combination of Bifidobacterium longum subsp. longum and Lacto-N-tetraose (LNT) for increasing serpin protein production in infants (<12 months) or young children (between one and less than three years).

WO2009/077352 describes prevention of opportunistic infections in immune-compromised individuals by combining various Bifidobacterium spp. with a fucosylated oligosaccharide, in infants (<12 months) or young children (between one and less than three years) and more particularly in premature and neo-natal infants.

### Objective

The inventors of the present disclosure have identified a novel way of increasing NMN and/or NAD+ in a subject in need thereof, which does not include ingestion of pharmaceuticals.

Further, the inventors of the present disclosure have recognized that there is a need to deliver certain health benefits related to nicotinamide adenine dinucleotide (NAD+) and/or nicotinamide mononucleotide (NMN) to subjects where the NAD+ and/or NMN does not have to be administered as such, but is rather produced in situ. Thus, NAD+ and/or NMN for example does not have to be ingested and pass through the acidic environment of the stomach. In the method of the present disclosures the NAD+ and/or NMN is produced directly in the gut or on the skin where it can easily be taken up by the body. Without being bound to any theory, the probiotic of the claimed combination/composition would become a member of the microbiota community, and as such, it would be able to stay longer in the gut while providing a prolonged and naturally controlled effect (i.e., limiting the risk of NAD precursor overdosage when ingested orally). Moreover, the combination of a prebiotic and a probiotic is easy to deliver, and well accepted by the end-consumers or health care practitioners. Further that there is also a need to deliver such benefits in a manner that does keep the cost of such delivery reasonable and affordable by most. Thus, the present disclosure describes compositions and methods that, when compared to existing compositions and methods, may be more efficient, more effective, and/or better suited for non-infants. In some embodiments, the compositions and methods of the present disclosure include one or more Bifidobacterium spp. with probiotic properties having a functional de novo nicotinamide adenine dinucleotide (nad) pathway that grow on at least one neutral fucosylated oligosaccharide (e.g., HMO) as disclosed herein. Components which are generally known to be safe to consume.

### SUMMARY

The present disclosure relates to a synbiotic combination which is capable of increasing the levels of nicotinamide mononucleotide (NMN) and/or nicotinamide adenine dinucleotide (NAD+) upon administration of the synbiotic combination, such as the intestinal/gut levels or the levels in the skin.

In one aspect of the disclosure, the synbiotic combination is a combination comprising a Bifidobacterium sp. capable of growing on a sialylated HMO and having a functional de novo nicotinamide adenine dinucleotide (nad) pathway and at least one fucosylated oligosaccharide for use in increasing nicotinamide mononucleotide (NMN) and/or nicotinamide adenine dinucleotide (NAD+) in the gut or in the skin of a subject when compared to a non-administered subject. The functional de novo nicotinamide adenine dinucleotide (nad) pathway of the Bifidobacterium sp. comprises the following functional genes nadC, nadD and nadE. Preferably the de novo nicotinamide adenine dinucleotide (nad) pathway is intact and in addition to the functional nad pathway it further comprises functional nadB and nadA genes.

In certain embodiments, a fucosylated oligosaccharide may be selected from the group consisting of 2′FL, 3FL, DFL, FSL LNFP-I, LNFP-II, LNFP-III, LNFP-V, LNFP-VI, LNDFH-I, LNDFH-II and LNDFH-III or a mixture thereof. Preferably, the fucosylated oligosaccharide is a fucosyllactose, such as 2′FL, 3FL or DFL, or a mixture thereof.

In various aspects, the Bifidobacterium sp. grow on one or more HMOs selected from the group consisting of neutral non-fucosylated HMOs, fucosylated HMOs, and sialylated HMOs. Preferably, the Bifidobacterium sp. can grow on one or more fucosylated HMOs, one or more neutral non-fucosylated HMOs, one or more sialylated HMOs, or combinations thereof, such as for example, on one or more HMOs selected from the group consisting of LNT, LNnT, LNT-II, 2′FL, 3FL, DFL, LNFP-I, 3′SL, 6′SL, and combinations thereof.

In one or more aspects, the Bifidobacterium sp. is selected from the group consisting of Bifidobacterium bifidum or Bifidobacterium longum or Bifidobacterium longum subsp. infantis. Preferably the Bifidobacterium bifidum species deposited as Bifidobacterium bifidum DSM 32403 and Bifidobacterium bifidum HA-132 (CNCM I-5898).

In further aspects of the disclosure, a synbiotic combination or composition as described herein is used in the treatment or prevention of a condition, disease or disorder by administering an effective amount of a synbiotic composition to a subject. Specifically, treatments or prevention related to mitochondria-related diseases, or a condition associated with altered mitochondrial function, age related symptoms or diseases, neurodegenerative diseases, cognitive disorders, reduced fertility, cardiovascular diseases, non-alcoholic fatty liver disease, inflammatory diseases, bacterial and viral infection, including sepsis caused by infections as well as UV induced skin damages such as photoaging and hyperpigmentation. In certain aspects, the ability of the synbiotic combination or composition to produce NMN and/or NAD+ in the gut or skin of the subject that provides the beneficial effect in any treatment or other use described herein when compared to a non-administered subject.

Furthermore, a synbiotic combination or composition as described herein may be used in the repair of damaged DNA or muscle regeneration or formation of beneficial short chain fatty acids in the gut or skin, in particular in infants.

Furthermore, a synbiotic combination or composition as described herein may be used to increase the abundance of the SCFA's acetate, propionate and/or butyrate in the gut of the subject, when compared to a non-administered subject.

In some embodiments, the subject may be a non-infant human such as a child, youth, or older adult or elderly individual.

In certain embodiments, the subject may by an infant (e.g., a human less than 3 years of age).

Features which are described in the context of separate aspects and embodiments of the invention may be used together and/or be interchangeable. Similarly, features described in the context of a single embodiment may also be provided separately or in any suitable subcombination.

### BRIEF DESCRIPTION OF THE DRAWINGS

FIG. 1 is an illustration of the NAD salvage pathway. Briefly: the salvage pathway involves NAD+ synthesis from its precursors, i.e., nicotinic acid (NA), nicotinamide (NAM) or nicotinamide riboside (NR). NA is catalytically converted to nicotinic acid mononucleotide (NAMN) by the action of nicotinic acid phosphoribosyltransferase (NAPT) and NAMN is converted to nicotinic acid adenine dinucleotide (NAAD) by the action of nicotinamide mononucleotide adenylyltransferase (NMNAT). NAM is converted by nicotinamide phosphoribosyl-transferase (NAMPT) to nicotinamide mononucleotide (NMN), which is also the product of phosphorylation of NR by nicotinamide riboside kinase (NRK) enzyme. Finally, NAAD is converted to NAD by the action of NAD synthase (NADS) enzymes, whereas NMN is converted to NAD by the NMNAT enzyme. Multiple enzymes break-down NAD+ to produce NAM and ADP-ribosyl moiety, however only sirtuins are depicted in this figure.

FIG. 2 is an illustration of the intact de novo nad pathway. Briefly: aspartate is converted to imonoaspartate by the action of a L-aspartate oxidase (encoded by nadB) which is converted to quinolinate by the action of a quinolinate synthase (encoded by nadA), which in turn is converted to nicotinic acid mononucleotide (NAMN stands for) by the action of acarboxylating nicotinate-nucleotide diphosphorylase (encoded by nadC) which is converted to stands for nicotinic acid adenine dinucleotide (NaAD) by the action of a nicotinate-nucleotide adenylyltransferase (encoded by nadD), which finally is converted to NAD+ by the action of a ammonia-dependent NAD(+) synthetase (encoded by nadE).

FIGS. 3A-3C are illustrations of bacterial growth curves obtained with BioLector for different probiotic strains with different carbon sources.

FIG. 3A is a chart of B. bifidum 32403 growth for the following carbon source, glucose, lactose or HMO mixture (2′FL, 2′FL: DFL, 3′SL, 6′SL, LNT and LNnT) (HMOs) and with no sugar as control.

FIG. 3B is a chart of L. reuteri DSM 12246 growth for lactose or HMO mixture (HMOs) and with no sugar as control.

FIG. 3C is a chart of L. plantarum TIFN101 growth for lactose or the following HMO mixture (HMOs) and with no sugar as control.

FIG. 4A-4E are illustrations of bacterial growth curves obtained with BioLector for different probiotic strains with different carbon sources

FIG. 4A is a chart of B. bifidum 32403 growth for the following carbon source: glucose, lactose, individual HMOs selected from 2′FL, 2′FL: DFL, 3′SL, 6′SL, LNT, LNnT and with no sugar as control.

FIG. 4B is a chart of B. bifidum HA-132 growth for the following carbon source: glucose, lactose, individual HMOs selected from 2′FL, 2′FL: DFL, 3FL, 3′SL, 6′SL, LNT, LNnT and with no sugar as control.

FIG. 4C is a chart of B. bifidum R0071 growth for the following carbon source: glucose, lactose, individual HMOs selected from 2′FL, 2′FL: DFL, 3FL, 3′SL, 6′SL, LNT, LNnT and with no sugar as control.

FIG. 4D is a chart of B. infantis HA-116 growth for the following carbon source: lactose, individual HMOs selected from 2′FL, 2′FL: DFL, 3FL, 3′SL, 6′SL, LNT, LNnT and with no sugar as control.

FIG. 4E is a chart of B. infantis R0033 growth for the following carbon source: lactose, individual HMOs selected from 2′FL, 2′FL: DFL, 3FL, 3′SL, 6′SL, LNT, LNnT and with no sugar as control.

FIG. 5A is a chart of metabolomic NMN results from supernatants from the seven strains B. bifidum 32403, L. reuteri DSM 12246, L. plantarum TIFN101, B. bifidum HA-132, B. bifidum R0071, B. infantis HA-116 and B. infantis R0033 grown on different carbon sources (i.e., Glucose, Lactose, Lactose+HMO mix, 2′FL, 2′FL: DFL, 3FL, 3′SL, 6′SL, LNT, LNnT) and with no sugar as control.

FIG. 5B is a chart of metabolomic NAD+ results from supernatants from the three strains B. bifidum 32403, L. reuteri DSM 12246, L. plantarum TIFN101, B. bifidum HA-132, B. bifidum R0071, B. infantis HA-116 and B. infantis R0033 grown on different carbon sources (i.e., Glucose, Lactose, Lactose+HMO mix, 2′FL, 2′FL: DFL, 3FL, 3′SL, 6′SL, LNT, LNnT) and with no sugar as control.

FIG. 6: Growth of B. bifidum 32403 on L-fucose or fucosyllactose sources with glucose and no sugar as control.

### DETAILED DESCRIPTION

The present disclosure relates to a synbiotic combination which is capable of increasing the intestinal/gut or skin levels of nicotinamide mononucleotide (NMN) and/or nicotinamide adenine dinucleotide (NAD+) upon administration of the sybiotic combination to a subject, when compared to a non-administered subject. A synbiotic combination is a beneficial combination of a probiotic (e.g., live microorganism(s) and a prebiotic (e.g., substrate(s) selectively used by host microorganism(s), that confers a health benefit on the host). Synbiotics may be complementary or synergistic. A ‘synergistic synbiotic’ is a synbiotic in which the substrate is designed to be selectively utilized by the co-administered microorganism(s). A ‘complementary synbiotic’ is a synbiotic composed of a probiotic combined with a prebiotic, which is designed to target autochthonous microorganisms that are resident in or colonize the host.

To our knowledge, it is the first time that a synbiotic composition has been found to be able to boost NMN and NAD+ levels via the microbiome such as the gut or skin microbiome. These results provide evidence that a synbiotic composition may further beneficially increase the systemic NMN and/or NAD+ pool in a subject in need thereof, such as for example a non-infant.

Nicotinamide adenine dinucleotide (NAD) is a coenzyme found in all living cells. It is a dinucleotide, consisting of two nucleotides joined through their phosphate groups. One nucleotide contains an adenine base, and the other contains nicotinamide. Enzymes such as the digestive enzymes in the stomach and small intestine cleave the NAD+ into nicotinamide mononucleotide (NMN), nicotinamide riboside (NR) and niacinamide (NAM) which are all absorbed from the intestine and capable of entering the cells of the subject.

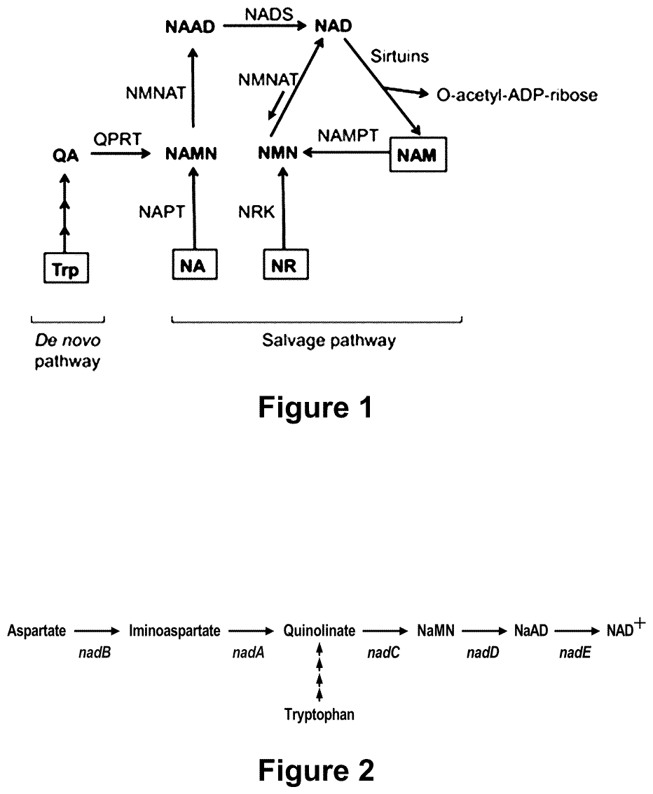

FIG. 1 is an illustration of the NAD+ salvage pathway, which provides cells with the ability to reuse NAD+ degradation products (NAM, NR and NMN) to regenerate NAD+. The salvage pathway converts niacinamide (NAM) to nicotinamide mononucleotide (NMN) using the enzyme nicotinamide phosphoribosyltransferase (NAMPT) or NMN is generated from nicotinamide riboside (NR) using the enzyme nicotinamide riboside kinase (NRK). NMN is then converted into NAD+ using the enzyme nicotinamide mononucleotide adenylyltransferase (NMNAT).

Nicotinamide mononucleotide (NMN) is the largest NAD+ precursor in the NAD+ salvage pathway. NMN is absorbed from the gut into blood circulation via an NMN transporter (Grozio et al. 2019 Nature Metabolism vol 1:47-57), where NMN is taken up by the cells and converted to NAD+. The advantage of using NMN as NAD precursor over the smaller precursor molecules NAM and NR, is that it requires less energy to convert it into NAD+ in the cell of the subject. NMN is currently used in dietary supplements to boost NAD+ production. Clinical trials have also been conducted with NMN which have concluded it is safe for oral administration in a dose of up to at least 500 mg, and that it is capable of increasing NAD+ concentrations (see for example Huang 2022 Frontiers in Ageing Volume 3, Article 851698).

NAD facilitates redox reactions, carrying electrons from one reaction to another. This means that NAD is found in two forms in the cell: in one form, NAD+ is an oxidizing agent that takes electrons from other molecules in order to become its reduced form, NADH. NAD+, and its reduced form NADH, are best known for their roles as coenzymes in redox reactions, linking the catabolic reactions of glycolysis and the TCA cycle to oxidative phosphorylation. NAD+ also has another role as a signaling molecule. From plants to metazoans, an increase in intracellular levels of NAD+ directs cells to make adjustments to ensure survival, including increasing energy production and utilization, boosting cellular repair, and coordinating circadian rhythms.

NAD+ levels are converted to signals by various enzymes that have evolved to sense NAD+, including the sirtuin deacylases (SIRT1-SIRT7), CtBPs, and poly-ADP-ribose polymerases (PARPs). They can sense NAD+ fluctuations because, unlike the enzymes of glycolysis and the TCA cycle, their dissociation constants for NAD+ are near physiological concentrations.

As a key substrate needed for respiration and other metabolic functions, NAD+ is an important molecule for studies involving healthy aging subjects. NAD+ has been observed to decline in organisms over time, in both aging and obese humans as well as in diabetic mice. Such an NAD+decline is a primary driver for the progression of biological dysfunction and age-related pathologies. Therefore, maintaining cellular NAD+ homeostasis may be an effective anti-aging strategy and a therapeutic option for improving the health lifespan and preventing aging-related symptoms or diseases.

Also, nicotinamide mononucleotide (NMN), a direct precursor of nicotinamide adenine dinucleotide (NAD+), may also be important due to significant anti-aging potential of nicotinamide phosphoribosyltransferase (NAMPT)/NAD+ signaling. As such NMN may be an effective anti-aging agent capable of extending the lifespan and ameliorating age-related symptoms or age-related complications.

NAD+ boosting drugs have been recently explored and are mostly NAD+ precursors (Baquero et al 2022, Frontiers in Mol. Biosci. vol 9 article 861603). As such, these NAD+ precursors can be supplemented, taken up and then converts into NAD+ inside the cell (e.g., via the NAD+ salvage pathway; FIG. 1). It has been demonstrated that an increase in NAD+ would be useful for the treatment of bacterial infections or for antibiotic treatment recovery (i.e., by facilitating microbiota recovery or by increasing fast-growing cells antibiotics uptake).

Other health benefits that may be achieved by NAD+ precursors are for example described in WO 2021/004922, which discloses treating or preventing a mitochondria-related disease or a condition associated with altered mitochondrial function in a subject in need thereof or at risk thereof.

In US 2022/0062158, purported benefits of yeast powder rich in NMN were reported to be useful for promoting antiinflammation, cardiovascular health, antioxidation, anti-aging, and/or relieving body fatigue.

A study by Jia et al., 2021 Front Physiol. 12:649547, describes broader effects of NMN that may include DNA damage inhibition, antiaging and anti-inflammatory effects, as well as hypoxia cellular damage reduction in unilateral ischemia-reperfusion injury (uIRI) mouse model.

Furthermore, an increase in NAD+ may be useful in reducing inflammation or symptoms thereof and it may also be useful in improving muscle repair or regeneration, in particular in a subject with a physical injury or accident, muscle immobilization, muscle overuse, loss of blood circulation, or lack of muscle use after injury (described in WO 2022/026612).

NAD+ may be a mediator of both antiviral and anti-inflammatory mechanisms. NAD+ may play an important role in fueling the activity of enzymes that regulate mammalian immune responses. PARPs and sirtuins are two NAD+-dependent enzyme families that participate in immune responses. By adding or removing post-translational modifications on key proteins such as nuclear factor kappa B (NF-κB), these enzymes can coordinate the intensity of inflammatory and immune responses. Thus, NAD+ may have an important position for both promoting strong immune responses to pathogens, and for keeping those responses in check. By increasing the activity of sirtuins, NAD+ may contribute to the resolution of inflammation, and to limiting or preventing the effects of cytokine storms.

This may be of particular importance in elderly, as aging may contribute to the disease scenario through reduced NAD+ and general dysregulation of the immune system, as evidenced by increased levels of inflammatory cytokines. The consequence is that NAD+ depletion may exacerbate the cytokine storm and lead to fatal outcome which is most common in older COVID-19 patients. Restoring normal NAD+ levels could decrease the severity of immune reaction in those patients and improve their clinical condition.

The term “Microbiota”, “microflora” and “microbiome” are used interchangeably and refer to a community of living microorganisms that typically inhabits a bodily organ or part in an animal or human. Particularly, in the gastrointestinal organs of animals or humans the microflora is termed the gastrointestinal or gut microbiome or microbiota. The most dominant members of the gastrointestinal microbiota in non-infant humans include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium, and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus, and Roseburia intestinalis. The gastrointestinal microbiota includes the mucosa-associated microbiota, which is located in or attached to the mucus layer covering the epithelium of the gastrointestinal tract, and luminal-associated microbiota, which is found in the lumen of the gastrointestinal tract.

The term “intestine” or “gut” are used interchangeably herein, and refers to the portion of the gastrointestinal tract consisting of the small intestine and the large intestine. The “large intestine” (Intestinum crassum) is the lower part of the gastrointestinal tract and is also referred to herein as “colon”.

The term ‘skin’ as used herein refers to the external surfaces of the human body including the oral cavity, the skin as well as the scalp. The term ‘skin microbiome’ as used herein refers to the group of microbes which colonize a defined skin area of an individual, such as e.g., the forehead, the forearm, the cheek or the scalp, without being limited thereto.

“Direct delivery” or “directly delivered” as used herein, means that the composition or an individual component of the composition is formulated in a manner such that the composition or component is not absorbed in the stomach and/or small intestine; rather the composition or component is made available in the distal intestinal tract, preferably the large intestine (colon), where it is available to or forms part of the microbiome.

### Probiotics

Without being bound by theory, it appears that in order for the at least one probiotic strain of the invention to increase nicotinamide mononucleotide (NMN) and/or nicotinamide adenine dinucleotide (NAD+) in the gut or the skin of a subject, it should: i) be able to grow on at least one sialylated HMO (preferably, on a selection of HMOs); ii) have a functional or intact de novo nad pathway; and iii) be fed with a fucosylated oligosaccharide, not just fucose or lactose, in order to produce NAD+. In particular, the inability to produce NAD+ when fed with fucose alone or when fucose was added as a supplement to a non-fucosylated carbon source which the cell can grow on, was surprising and indicates that the oligosaccharide form of the fucose is important for the probiotic to produce NAD+.

In the context of the present disclosure, the term “Probiotic” refers to microbial cells or cell preparations, such as bacteria, which, when ingested in adequate amounts, provide a benefit to the host (human or animal) by replenishing or otherwise supplementing the natural gastrointestinal flora or by eliminating undesired bacteria in the gastrointestinal (GI) tract or by executing beneficial metabolic activities along the GI tract or by stimulating the immune system. In various aspects of the present disclosure, the disclosed probiotic is a bacterium which can be used alone or in combination with another disclosed probiotic bacterium. In an embodiment, the disclosed probiotic is selected from the Bifidobacterium genus. In another embodiment, the disclosed probiotic belonging to the Bifidobacterium genus is selected from, without being limited to, the following Bifidobacterium sp.: B. adolescentis, B. angulatum, B. animalis, B. animalis subsp. animalis, B. animalis subsp. lactis, B. asteroides, B. biavatii, B. bifidum, B. breve, B. catenulatum, B. coagulans, B. longum, B. infantis, B. longum subsp. infantis, B. longum subsp. longum, B. magnum, B. coryneforme, B. dentium, B. gallicum, or B. subtile. In some another embodiment, the probiotic can be selected from the group consisting of B. bifidum or B. longum. In yet another embodiment, the probiotic is selected from the group consisting of B. bifidum or B. longum subsp. infantis. In a further embodiment, the Bifidobacterium sp. is a Bifidobacterium bifidum, for example, the deposited Bifidobacterium bifidum DSM 32403 or Bifidobacterium bifidum HA-132 (CNCM I-5898).

In one embodiment, the disclosed probiotic is a Bifidobacterium sp. capable of growing on a broad selection of HMOs since Bifidobacterium spp. that do not produce NAD+ only grow on a limited selection of HMOs. In an embodiment, the Bifidobacterium sp. may grow on one or more HMOs selected from the group consisting of neutral non-fucosylated HMOs, neutral fucosylated HMOs and sialylated HMOs. In another embodiment, the Bifidobacterium sp. can grow on one or more of neutral non-fucosylated HMOs such as, without being limited to, LNT-II, LNT or LNnT; neutral fucosylated HMOs such as, without being limited to, 2′FL, 3FL or DFL; and sialylated HMOs such as, without being limited to, 3′SL, 6′SL, FSL, LST-a, LST-B, or LST-c). In yet another embodiment, the Bifidobacterium sp. can grow on any one of the neutral non-fucosylated HMOs (such as LNT-II, LNT or LNnT), fucosylated neutral HMOs (such as 2′FL, 3FL or DFL) and sialylated HMOs (such as 3′SL or 6′SL). In some embodiments, the Bifidobacterium sp. can grow on at least one neutral HMO, such as an HMO selected from the group consisting of LNT, LNnT, 2′FL, 3FL and DFL. In some another embodiment, the Bifidobacterium sp. can grow on at least one neutral non-fucosylated HMO and at least one neutral fucosylated HMO's. In a further embodiment, the Bifidobacterium sp., can grow on at least one fucosyllactose, such as 2′FL, 3FL and/or DFL. In some specific embodiment, the Bifidobacterium sp. can grow on 3′SL and/or 6′SL. In a further embodiment, the Bifidobacterium sp. can grow on 3′SL and/or 6′SL and one or more HMOs selected from the group consisting of LNT, LNnT, 2′FL, 3FL and DFL.

Interestingly, the inventors of the present application observed that despite the ability to grow on any of the above-mentioned HMO's the Bifidobacterium bifidum 32403 and Bifidobacterium bifidum HA-132 (CNCM I-5898) were only observed to produce NAD+ and/or NMN above the level produced when grown on lactose when feeding on a fucosylated oligosaccharide, in particular a fucosyllactose. Moreover, the same increase in NAD+ and/or NMN was not observed with neutral core HMOs or sialylated HMOs, nor with lactose or fucose as carbon sources.

When compared to two probiotic strains Lactobacillus reuteri DSM 12246 and L. plantarum TIFN101, which were only observed to grow on lactose and which were not observed to produce NAD+ or NMN) it became apparent that such Lactobacillus strains do not have an intact de novo nad pathway.

Furthermore, it was found that Bifidobacterium sp., which are not able to grow on sialylated HMOs are not capable of producing NAD+ or NMN. In one embodiment, the disclosed probiotic is a Bifidobacterium species having a functional de novo nicotinamide adenine dinucleotide (nad) pathway. In the context of the present disclosure, the expression “functional de novo nad pathway” refers to the ability of producing NAD+ from quinolinate which requires the presence of the functional genes nadC, nadD and nadE in the genome of the probiotic strain. A functional gene as described herein refers to a wild type gene or a functional variant thereof which is capable of converting the desired substrate (e.g., quinolinate) into a desired product (e.g., nicotinic acid mononucleotide (NAMN)) inside the cell or in an appropriate in-vitro assay. In a preferred embodiment an example of a functional gene as described herein is one found in Bifidobacterium bifidum 32403 and Bifidobacterium bifidum HA-132 (CNCM I-5898). A functional variant of a gene or a polypeptide as described herein is a protein/nucleic acid sequence with alterations in the genetic code, which retain its original functionality. A functional variant may be obtained by mutagenesis or may be natural occurring variants from the same or other species. The functional homologue should have a remaining functionality/activity of at least 50%, such as at least 60%, 70%, 80%, 90% or 100% compared to the functionality of the original protein/nucleic acid sequence.

The quinolinate can be produced via the kynurenine pathway starting from tryptophan (FIG. 1) or can be produced from aspartate using the enzymes L-aspartate oxidase (nadB) and quinolinate synthase (nadA). In some embodiment, the disclosed probiotic is a Bifidobacterium species which comprises the nadB and nadA genes in addition to functional de novo nicotinamide adenine dinucleotide (nad) pathway (nadC, nadD and nadE genes). In some another embodiment, the disclosed probiotic is a Bifidobacterium species having an intact de novo nad pathway. In the context of the present disclosure, the expression “intact de novo nad pathway” refers to the ability of producing NAD+ from aspartate which requires the presence of the functional genes, nadB, nadA, nadC, nadD and nadE in the genome of the probiotic strain (FIG. 2). Still in the context of the present disclosure, nadB, nadA and nadC are preferably encoded from a single operon whereas nadD and nadE are in separate areas of the genome and are not part of the nadBAC operon. The presence of a functional and/or intact de novo nad pathway can be identified by sequencing, either by whole genome sequencing or by sequencing using primers specific for the individual nadB, nadA, nadC, nadD and nadE genes in the pathway as disclosed herein and/or as may be known to persons of ordinary skill in the art.

It may be noted that enzymes with the indicated activity and which are part of the de novo nad pathway may be identified by a person skilled in the art as illustrated via the following examples. NadB encodes for a polypeptide having L-aspartate oxidase activity (e.g., as seen in NCBI ref seq: WP_013389689), which converts aspartate to iminoaspartate. NadA encodes for a polypeptide having quinolinate synthase activity (see e.g., NCBI ref seq: WP_013389688) which converts iminoaspartate to quinolinate. As an alternative quinolinate can be formed from tryptophan via the kynurenine pathway. NadC encodes for a polypeptide having carboxylating nicotinate-nucleotide diphosphorylase activity (see e.g., NCBI ref seq: WP_003812421) which converts quinolinate to nicotinic acid mononucleotide (NaMN). NadD encodes for a polypeptide having nicotinate-nucleotide adenylyltransferase activity (see e.g., NCBI ref seq: WP_226800914) which converts NaMN to nicotinic acid adenine dinucleotide (NaAD). NadE encodes for a polypeptide having ammonia-dependent NAD+ synthetase activity (see e.g., NCBI ref seq: WP_013390069.1) which converts NaAD to nicotinamide adenine dinucleotide (NAD+). It may be noted that different probiotic bacteria may have functional homologues or functional variants of the above-mentioned enzymes in the de novo nad pathway. In the context of the present disclosure, a functional homologue or functional variant of a protein/nucleic acid sequence as described herein is a protein/nucleic acid sequence with alterations (i.e., mutations, deletions, additions) compared to the original sequence, while retaining its original functionality. A functional homologue may be obtained by mutagenesis or may be natural occurring variants from the same or other species. The functional homologue should have a remaining functionality/activity of at least 50%, such as at least 60%, 70%, 80%, 90%, 91%, 92, 93, 94, 95, 96, 97, 98, 99 or 100% compared to the functionality of the protein/nucleic acid sequence. Accordingly, it may be noted that the NCBI reference numbers above are only illustrative examples of how probiotic species of Bifidobacterium with functional genes encoding certain polypeptides having an enzymatic activity products may be utilized in accordance with the compositions and methods disclosed herein. Preferably, the de novo nad pathway genes are native genes of a Bifidobacterium sp. The homology of the polypeptides having a L-aspartate oxidase activity is approximately 75-100% across the Bifidobacterium bifidum and Bifidobacterium longum. The homology of the polypeptides having a quinolinate synthase activity is approximately 75-100% across the Bifidobacterium bifidum and Bifidobacterium longum. The homology of the polypeptides having a carboxylating nicotinate-nucleotide diphosphorylase activity is approximately 75-100% across the Bifidobacterium bifidum and Bifidobacterium longum. The homology of the polypeptides having a nicotinate-nucleotide adenylyltransferase activity is approximately 75-100% across the Bifidobacterium bifidum and Bifidobacterium longum. The homology of the polypeptides having an ammonia-dependent NAD(+) synthetase is approximately 75-100% across the Bifidobacterium bifidum and Bifidobacterium longum. In one embodiment, the homology of the polypeptides having the listed enzymatic activities (i.e., L-aspartate oxidase, quinolinate synthase, carboxylating nicotinate-nucleotide diphosphorylase, nicotinate-nucleotide adenylyltransferase, and/or ammonia-dependent NAD(+) synthetase activities) is between 75 and 100% across the Bifidobacterium bifidum and Bifidobacterium longum species. In an embodiment, the homology of the polypeptides having the listed enzymatic activities is of at least 75, 76, 77, 78, 79, 80, 81, 82, 83, 84, 85, 86, 87, 88, 89, 90, 91, 92, 93, 94, 95, 96, 97, 98, 99, or 100% across the Bifidobacterium bifidum and Bifidobacterium longum species. In another embodiment, the homology of the polypeptides having the listed enzymatic activities is no more than 100, 99, 98, 97, 96, 95, 94, 93, 92, 91, 90, 89, 88, 87, 86, 85, 84, 83, 82, 81, 80, 79, 78, 77, 76, or 75% across the Bifidobacterium bifidum and Bifidobacterium longum species. In yet another embodiment, the homology of the polypeptides having the listed enzymatic activities is between 75, 76, 77, 78, 79, 80, 81, 82, 83, 84, 85, 86, 87, 88, 89, 90, 91, 92, 93, 94, 95, 96, 97, 98, 99, or 100% and 100, 99, 98, 97, 96, 95, 94, 93, 92, 91, 90, 89, 88, 87, 86, 85, 84, 83, 82, 81, 80, 79, 78, 77, 76, or 75% across the Bifidobacterium bifidum and Bifidobacterium longum species.

An aspect of the present disclosure is a synbiotic combination wherein the probiotic part of the combination is at least one Bifidobacterium sp. capable of growing on a sialylated HMO and having a functional de novo nicotinamide adenine dinucleotide (nad) pathway.

In a preferred embodiment, the at least one Bifidobacterium sp. has an intact de novo nad pathway and/or is capable of growing on one or more HMOs selected from the group consisting of a neutral core HMO, a fucosylated HMO and a sialylated HMO.

In one embodiment of the disclosure, the at least one Bifidobacterium sp. capable of growing on a sialylated HMO and having a functional de novo nicotinamide adenine dinucleotide (nad) pathway is capable of producing NAD+ and/or NMN when cultivated in the presence of a fucosylated oligosaccharide, in particular a fucosylated HMO selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof. In a preferred embodiment the fucosylated oligosaccharide is a fucosyllactose, such as 2′FL, 3FL, or DFL, or a mixture thereof.

In a further embodiment, the probiotic cells of the present disclosure may be deficient in or have reduced nicotinamide mononucleotide adenylyltransferase activity, which allows for the accumulation of NMN. In particular, if at least one of the polypeptides having nicotinamide phosphoribosyltransferase (NAMPT, e.g., a functional homologue of NCBI accession nr. WP_047288731) or nicotinamide riboside kinase (NRK) activity are functional allowing formation of NMN from NR and NAM.

In one embodiment, the at least one Bifidobacterium sp. i) has a functional or intact de novo nicotinamide adenine dinucleotide (nad) pathway, and ii) is deficient in or have reduced activity of the nicotinamide mononucleotide adenylyltransferase enzyme, and iii) has at least one functional enzyme selected from nicotinamide phosphoribosyltransferase (NAMPT) or nicotinamide riboside kinase (NRK). Preferably, both NAMPT and NRK are functional.

In a further embodiment, the at least one Bifidobacterium sp. has the following characteristics i) has a functional or intact de novo nicotinamide adenine dinucleotide (nad) pathway, ii) is deficient in or have reduced activity of the nicotinamide mononucleotide adenylyltransferase enzyme, iii) has at least one functional enzyme selected from nicotinamide phosphoribosyltransferase (NAMPT) or nicotinamide riboside kinase (NRK) and, iv) is capable of growing on a fucosylated HMO. Preferably, the at least one Bifidobacterium sp. is capable of growing on at least one additional HMO selected from neutral non-fucosylated HMOs or sialylated HMOs.

### Prebiotics

“Prebiotic” is a term used to describe compounds in food that induce the growth or activity of beneficial microorganisms such as bacteria and fungi in the gut of an animal species or a human. In the gastrointestinal tract, prebiotics can alter the composition of organisms in the gut microbiome. Dietary prebiotics are typically nondigestible fiber compounds that pass undigested through the upper part of the gastrointestinal tract and stimulate the growth or activity of advantageous bacteria in the colon by acting as substrates for them. Common prebiotics used in food manufacturing include beta-glucan from oats and inulin from chicory root. Oligosaccharides that are undigestible by humans and animals, like fructo-oligosaccharides (FOS) and galacto-oligosaccharides (GOS) and certain human milk oligosaccharides (HMOs) may act as prebiotics for certain bacterial species.

In the context of the disclosure, the term “oligosaccharide” means a saccharide polymer containing a number of monosaccharide units. In some embodiments, preferred oligosaccharides are saccharide polymers consisting of three to nine monosaccharide units, preferred oligosaccharides are tri-saccharides, tetra-saccharides, penta-saccharides or hexa-saccharides. In relation to the present disclosure the oligosaccharides are fucosylated oligosaccharides, preferably a neutral fucosylated human milk oligosaccharide.

The term “human milk oligosaccharide” or “HMO” in the present context refers to a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an alpha-L-fucopyranosyl and/or an alpha-N-acetyl-neuraminyl (sialyl) moiety. HMO structures are e.g., disclosed by Xi Chen in Chapter 4 of Advances in Carbohydrate Chemistry and Biochemistry 2015 vol 72.

In the context of the present disclosure, lactose (a disaccharide) is not regarded as an HMO species.

HMOs can be non-acidic (or neutral) or acidic. Neutral HMOs are devoid of a sialyl residue and acidic have at least one sialyl residue in their structure. The non-acidic (or neutral) HMOs can be fucosylated or non-fucosylated.

Examples of such neutral non-fucosylated (neutral core) HMOs include lacto-N-triose 2 (LNT-2) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

Examples of neutral fucosylated HMOs include 2′-fucosyllactose (2′FL), lacto-N-fucopentaose I (LNFP-I), lacto-N-difucohexaose I (LNDFH-I), 3-fucosyllactose (3FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH).

Examples of acidic HMOs include 3′-sialyllactose (3′SL), 6′-sialyllactose (6′SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

The production of various fucosylated oligosaccharides and certain particular HMOs is well known. See for example Bych et al 2019 Current Opinion in Biotechnology 56:130-137 for a review on HMO production. In a preferred embodiment, a fucosylated oligosaccharide, such as the fucosylated HMO of the present disclosure may be produced synthetically meaning it is produced ex vivo chemically and/or biologically, e.g., by means of chemical reaction, enzymatic reaction or from recombinant cell cultures. For example, various fucosylated oligosaccharides can be made as described in WO2012/127410, WO 2010/115934, WO 2010/115935, WO 2013/139344 or PCT/EP2021/086932.

In one or more preferred embodiments, the one or more fucosylated oligosaccharide(s) is a fucosylated HMO, preferably selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof.

In one embodiment, the fucosylated oligosaccharide is a fucosyllactose, such as 2′FL, 3FL, DFL, FSL, preferably a neutral fucosyllactose such as 2′FL, 3FL and/or DFL or a mixture thereof. The present disclosure relates to a synbiotic composition comprising a probiotic having a functional de novo nicotinamide adenine dinucleotide (nad) pathway and at least one fucosylated oligosaccharide.

### Synbiotic Combination/Composition

An aspect of the present disclosure is a composition comprising at least one Bifidobacterium sp. capable of growing on a sialylated HMO and having a functional de novo nicotinamide adenine dinucleotide (nad) pathway and at least one neutral fucosylated HMO. In a preferred embodiment, the at least one Bifidobacterium sp. has an intact de novo nad pathway and/or is capable of growing on one or more HMOs selected from the group consisting of neutral non-fucosylated HMOs and neutral fucosylated HMOs.

In one embodiment, the sialylated HMO is 3′SL, 6′SL, FSL, LST-a, LST-b, LST-c. In preferred embodiments the sialylated HMO is sialyllactose, such as 3′SL and 6′SL.

In one embodiment, the fucosylated HMO in the composition of the present disclosure is selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof. Preferably, the fucosylated HMO is a fucosyllactose, preferably selected from 2′FL, 3FL, DFL, or a mixture thereof.

Exemplary Bifidobacterium sp. of the synbiotic compositions are described in the section “probiotics”.

In one embodiment, the composition of the present disclosure comprises: a) at least one Bifidobacterium sp. that has: i) a functional or intact de novo nicotinamide adenine dinucleotide (nad) pathway, and ii) is capable of growing on a sialylated HMO and a fucosylated HMO. In such an embodiment, the composition further includes b) a fucosylated HMO selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof. Preferably, the at least one Bifidobacterium sp. is capable of growing on at least one additional HMO selected from neutral non-fucosylated HMOs.

In one embodiment, the composition of the present disclosure comprises a) at least one Bifidobacterium sp. that has i) a functional or intact de novo nicotinamide adenine dinucleotide (nad) pathway, and ii) is capable of growing on a sialylated HMO and on a fucosylated HMO and b) a fucosylated oligosaccharide in the composition of the present invention is selected from the group consisting of 2′FL, 3FL, DFL, or a mixture thereof. Preferably, the at least one Bifidobacterium sp. is capable of growing on at least one additional HMO selected from neutral non-fucosylated HMOs, such as for example, one or more HMOs selected from the group consisting of LNT-II, LNT, LNnT.

In one embodiment, the composition of the present disclosure comprises: a) at least one Bifidobacterium sp. that: has i) a functional or intact de novo nicotinamide adenine dinucleotide (nad) pathway, and ii) is deficient in or have reduced activity of the nicotinamide mononucleotide adenylyltransferase enzyme; and iii) has at least one functional enzyme selected from nicotinamide phosphoribosyltransferase (NAMPT) or nicotinamide riboside kinase (NRK). In such an embodiment, the composition further comprises b) a neutral fucosylated HMO selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof. Preferably, both NAMPT and NRK are functional.

In one embodiment, the composition of the present disclosure comprises: a) at least one Bifidobacterium sp. that has i) a functional or intact de novo nicotinamide adenine dinucleotide (nad) pathway, and ii) is deficient in or have reduced activity of the nicotinamide mononucleotide adenylyltransferase enzyme; and iii) has at least one functional enzyme selected from nicotinamide phosphoribosyltransferase (NAMPT) or nicotinamide riboside kinase (NRK). In such an embodiment, the composition further comprises b) a neutral fucosylated oligosaccharide in the composition of the present invention is selected from the group consisting of 2′FL, 3FL, DFL or a mixture thereof.

In a further embodiment, the composition of the present disclosure comprises a) at least one Bifidobacterium sp. that has the following characteristics: i) has a functional or intact de novo nicotinamide adenine dinucleotide (nad) pathway, and ii) is deficient in or has reduced activity of the nicotinamide mononucleotide adenylyltransferase enzyme, and iii) has at least one functional enzyme selected from nicotinamide phosphoribosyltransferase (NAMPT); and iv) is capable of growing on a sialylated HMO and a fucosylated HMO. In such an embodiment, the composition further comprises a neutral fucosylated HMO selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof. Preferably, the at least one Bifidobacterium sp. is capable of growing on at least one additional HMO selected from neutral core HMOs or sialylated HMOs, such as any one of the HMOs selected from the group consisting of LNT, LNnT, 3′SL and 6′SL.

In a further embodiment, the composition of the present disclosure comprises a) at least one Bifidobacterium sp., which has the following characteristics: i) has a functional or intact de novo nicotinamide adenine dinucleotide (nad) pathway, and ii) is deficient in or have reduced activity of the nicotinamide mononucleotide adenylyltransferase enzyme, and iii) has at least one functional enzyme selected from nicotinamide phosphoribosyltransferase (NAMPT); and iv) is capable of growing on a sialylated HMO and a fucosylated HMO and b) a neutral fucosylated oligosaccharide selected from the group consisting of 2′FL, 3FL, DFL, or a mixture thereof.

In one embodiment, the at least one Bifidobacterium sp. in the composition of the present disclosure is selected from the group consisting of Bifidobacterium Bifidum or Bifidobacterium longum. In another embodiment, the at least one Bifidobacterium sp. in the composition of the present disclosure is selected from the group consisting of Bifidobacterium Bifidum or Bifidobacterium longum subsp. infantis. More preferably the at least one Bifidobacterium sp. is Bifidobacterium bifidum is Bifidobacterium bifidum DSM 32403 or Bifidobacterium bifidum HA-132 (CNCM I-5898).

In one embodiment, the at least one Bifidobacterium sp. in the composition of the present disclosure is capable of producing more NMN and/or NAD+ when growing on fucosyllactose (2′FL, 2′FL: DFL or 3FL) than when growing on non-fucosylated neutral HMOs (i.e., LNT and LNnT) or on sialylated HMOs (i.e., 3′SL and 6′SL). In an embodiment, the NMN and/or NAD+production of the at least one Bifidobacterium sp. is of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 or more fold more effective when grown on fucosyllactose compared to when grown on non-fucosylated neutral HMOs or on sialylated HMOs.

The composition may be provided as a powder, a dry composition, a suspension, a liquid concentrate, an emulsion or a gel comprising a mixture of the probiotics and the fucosylated HMO. The formulation may be a ready to use formulation, such as a unit dosage form, i.e., a capsule, tablet or sachet/stick pack or a formulation that needs to be dissolved in a liquid prior to use. The composition may also be in the form of a kit of parts with the probiotic in one compartment and the fucosylated oligosaccharide in another compartment and an instruction describing the best intake form.

For topical application, it is well understood that such compositions comprise a physiologically acceptable medium, i.e. a medium compatible with keratinous substances, such as the skin, mucous membranes, and keratinous fibres. In particular, the physiologically acceptable medium is a cosmetically acceptable carrier which is carriers and/or excipients and/or diluents conventionally used in topical cosmetic compositions such as in particular in skin care preparations. In addition, the acceptable carrier being a part of the composition for topical administration is foreseen to be water.

In particular, the topic compositions according to the present invention are cosmetic or pharmaceutical compositions, preferably cosmetic (non-therapeutic) compositions. The term “cosmetic composition” as used in the present application refers to cosmetic compositions as defined under the heading “Kosmetika” in Römpp Lexikon Chemie, 10^{th} edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

Preferred compositions according to the invention are skin care preparations, decorative preparations, and functional preparations. Examples of skin care preparations are, in particular, light protective preparations, anti-aging preparations, preparations for the treatment of photo-aging, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations. Examples of decorative preparations are, in particular, lipsticks, eye shadows, mascaras, dry and moist make-up formulations, rouges and/or powders. Examples of functional preparations are cosmetic or pharmaceutical compositions containing active ingredients such as hormone preparations, vitamin preparations, vegetable extract preparations, anti-aging preparations, and/or antimicrobial (antibacterial or antifungal) preparations without being limited thereto.

The synbiotic composition can also be in a nutritional composition, such as a medical nutrition product. It can contain sources of protein, lipids, vitamins, minerals and/or digestible carbohydrates and can be in powdered or liquid forms. The composition can be designed to be the sole source of nutrition or a nutritional supplement.

### Use of the Synbiotic Combination/Composition

The present disclosure discloses one or more methods of treating a condition, disease, or disorder, or of providing a subject with a health benefit by administering an effective amount of a synbiotic composition which is capable of increasing the levels of nicotinamide mononucleotide (NMN) and/or nicotinamide adenine dinucleotide (NAD+) in the intestine/gut or in the skin of said subject when compared to a non-administered subject.

The term ‘treatment’ as used herein refers to both treatment of an existing disease (e.g., a disease or disorder as herein referred to), or prevention of a disease, i.e. prophylaxis. It will therefore be recognized that treatment as referred to herein may, in some embodiments, be prophylactic.

Furthermore, the present disclosure provides a non-medical use of an effective amount of a synbiotic composition which is capable of increasing the levels of nicotinamide mononucleotide (NMN) and/or nicotinamide adenine dinucleotide (NAD+) in the intestine/gut or in the skin of a subject when compared to a non-administered subject. In particular, such non-medical use can be in a dietary supplement or a cosmetic (non-therapeutic) treatment.

An aspect of the present disclosure is a combination comprising at least one Bifidobacterium sp. capable of growing on a sialylated HMO and having a functional de novo nicotinamide adenine dinucleotide (nad) pathway and at least one neutral fucosylated oligosaccharide for use in increasing NMN and/or NAD+ in the gut and/or the skin of a subject when compared to a non-administered subject.

The synbiotic combination for the use according to the present disclosure can be a composition as described in the section “synbiotic composition”. As the term combination indicates the two components, namely the at least one Bifidobacterium sp. having a functional de novo nicotinamide adenine dinucleotide (nad) pathway or an intact nicotinamide adenine dinucleotide (nad) pathway and the fucosylated oligosaccharide may also be formulated as two separate compounds (e.g., a kit of parts) which can either be mixed prior to the administration and administered simultaneously. Alternatively, one compound may be administered before the second compound. For example, the probiotic can be administered 1 to 5 hours before the fucosylated oligosaccharide (e.g., neutral fucosylated HMO) to allow the probiotic to start propagating in the gut or the skin prior to administering the neutral fucosylated HMO.

### Mitochondria-Related Disease

In one aspect of the present disclosure, the synbiotic combination and/or composition of the present disclosure is used for treating or preventing a mitochondria-related disease or a condition associated with altered mitochondrial function, in a subject in need thereof or at risk of having a mitochondria-related disease. Preferably, the NMN and/or NAD+ levels are increased in one or more cells of the subject when compared to a non-administered subject, for example one or more cells that are part of at least one body part selected from the group consisting of gut, liver, heart, eye, kidney, reproductive organs, such as ovaries, neurons, brain, and muscle, such as skeletal muscle. In some embodiments, the composition is administered to a non-infant such as an older adult or an elderly individual.

The mitochondria-related disease or condition can be selected from the group consisting of deleterious effects of aging, stress, obesity, overweight, reduced metabolic rate, metabolic syndrome, diabetes mellitus, complications from diabetes, hyperlipidemia, neurodegenerative disease, cognitive disorder, stress-induced or stress-related cognitive dysfunction, mood disorder, anxiety disorder, age-related neuronal death or dysfunction, acute kidney injury (AKI), chronic kidney disease (CKD), kidney failure, trauma, infection, hearing loss, macular degeneration, myopathies and dystrophies, and combinations thereof.

A number of these mitochondria-related disease are also related to aging as described in the aging section below.

### Aging

NAD+ levels are steadily decline during aging. By the time a mouse or human is middle aged, levels of NAD+ have fallen to half of youthful levels, with resulting loss of sirtuin and PARP activity.

For example, aging is a condition that can be linked to one of the following: oxidative stress, reduced level of glutathione, and lower redox ratio NAD+/NADH, and increase in CD38 leading to NAD+-consumption, hence CD38 is an enzyme responsible for the age-related NAD+ decline. The compositions disclosed herein can treat or prevent these deleterious effects of aging. For example, increased NAD+ may lead to enhancement of glutathione through redox recycling. As other examples, depression is linked to low glutathione, and anxiety is linked to oxidative stress.

An aspect of the present disclosure is a method or use of a synbiotic combination and/or composition of the present invention for increasing NMN and/or NAD+ by administering an effective amount of the synbiotic combination and/or composition to non-infant subjects to slow down its aging process, extending its lifespan and vitality when compared to a non-administered non-infant subject.

The increase in NMN and/or NAD+ provided by the combinations/compositions of the present disclosure may be used to treat, prevent, or improve conditions associated with aging when compared to a non-administered subject. In particular, improving insulin sensitivity in the elderly may be an objective of the combinations/compositions of the present disclosure including treating, age-associated loss of insulin sensitivity in a subject (WO2014/146044). Another beneficial use of the combinations/compositions of the disclosure herein is in the treatment of skin ageing including photoaging.

NMN was shown to prevent aging-induced cognitive impairment by improving cerebrovascular and mitochondrial function and reducing apoptosis in the prefrontal cortex and hippocampus of aged animals (Hosseini et al 2019 Neuroscience, 423:29-37).

Death of photoreceptors results in vision loss, and this occurs in many diseases, including age-related macular degeneration (AMD). Retinal NAD+ deficiency was observed in multiple mouse models with retinal dysfunction, including light-induced degeneration, streptozotocin (STZ)-induced diabetic retinopathy, and aging-associated retinal dysfunction. NAD+ deficiency in photoreceptors causes significant glycolytic and mitochondrial dysfunction under basal conditions and impairs the normal response to moderate metabolic stresses, which results in photoreceptor cell death and retinal degeneration. Mills et al. 2016 Cell Metab 24 (6): 795-806 reported that long-term NMN treatment via biochemical administration ameliorated age-associated pathological changes in the eyes and prevented rod cell dysfunction in aged C57BL/6N mice. Moreover, NMN treatment via biochemical administration suppressed age-associated bone density loss, enhanced energy metabolism, promoted physical activity, improved insulin sensitivity and plasma lipid profile, and enhanced mitochondrial respiratory capacity in skeletal muscle. Furthermore, administration via the disclosed synbiotic combinations and method means that the NAD+ and/or NNM does not have to pass through the acidic environment of the stomach and instead is produced directly in the gut where it can easily be taken up by the body.

In some embodiments, the combination and/or composition of the present disclosure, which synbiotically increase NMN and/or NAD+ levels in a subject when compared to a non-administered subject, may be used for prevention or delaying the onset of age-related conditions and diseases where aging is a causal factor, such as: age-induced cognition impairment, age-related memory disorder, macular degeneration, vision loss associated with retinal degeneration, cardiovascular diseases, atherosclerosis, gastrointestinal diseases, constipation, diabetes, loss of insulin sensitivity, loss of vitality, reduction of muscle mass and endurance, reduction of bone density, declined immune function, susceptibility to infectious diseases.

### Metabolism and Diabetes

Impaired NAD+-mediated sirtuin signaling is also implicated in insulin resistance and type 2 diabetes (T2DM). In particular, defective SIRT1 activity is thought to be a factor in impaired insulin sensitivity. Although an increase in NAD+ and or NMN via oral delivery may be useful for age-associated loss of insulin sensitivity (WO2014/146044), administration via the disclosed synbiotic combinations and methods provides for NAD+/NMN to be produced directly in the gut where it can readily be made available to other portions of body rather than the NAD+/NMN having the to pass through the esophagus and stomach.

In an aspect of the present disclosure, the synbiotic combination and/or composition may be used for increasing NAD+ and/or NMN levels of a subject when compared to a non-administered subject while treating obesity, overweight, reduced metabolic rate, insulin resistance, metabolic syndrome, type 2 diabetes mellitus and complications from diabetes.

### Kidney Related Diseases

In some embodiments, the combination and/or composition of the present disclosure, which synbiotically increase NMN and/or NAD+ of a subject when compared to a non-administered subject, is useful for prevention and/or amelioration of acute kidney injury (AKI), chronic kidney disease (CKD), and/or kidney failure.

Thus, in a further embodiment, the increase in NMN and/or NAD+ provided by the combinations/compositions of the disclosure may be advantageously used to treat or ameliorate acute kidney injury (AKI), chronic kidney disease (CKD) and/or kidney failure.

### Gastrointestinal Motility

NAD+ depleting drugs cause various gastrointestinal symptoms, including constipation, suggesting NAD+ may play a role in regulating colon function. Results from a mouse study by Zhu et al 2017 Signal Transduct Target Ther. 2:17017, observed that repletion of NAD+ attenuated several colonic ageing phenotypes and improved defecation in old mice. One possible mechanism is that NAD+ serves as an enteric inhibitory neurotransmitter thereby contributing to neural regulation of colonic motility. Susceptibility to constipation may increase with age, coinciding with the age-related decreasing NAD+ levels.

Accordingly, in one or more aspects of the present disclosure, an increase in NMN and/or NAD+provided by the administration of the synbiotic combination and/or composition of the present disclosure may be used for treating or preventing constipation.

### Fertility

NMN is highly relevant in the context of infertility owing to its various anti-aging effects and is used by IVF patients as a supplement. For example, long-term treatment with nicotinamide mononucleotide (NMN) may be effective to improve age-related diminished ovary reserve through enhancing the mitophagy level of granulosa cells in mice (Huang et al 2022 J Nutr Biochem 101:108911). As a further example, multigenerational obesity-induced perturbations in oocyte-secreted factor signaling may be ameliorated by NMN treatment (Bertoldo et al. 2018 Human Reproduction Open, pp. 1-13). However, administration via the disclosed synbiotic combinations and method allows the NAD+/NMN to be produced directly in the gut without being directly exposed to or absorbed by the stomach or esophagus. In this context, the increase in NMN and/or NAD+ provided by the synbiotic combinations/compositions of the invention may provide improvements in fertility treatment of obese women.

In a further aspect, the increase in NMN and/or NAD+ provided by the synbiotic combinations/compositions of the invention may be used to treat or improve age-related diminished ovary reserve and may be effective against ovarian aging when compared to a non-administered subject.

### Liver Related Diseases

Sufficient NAD+ levels are essential for adequate mitochondrial fatty acid oxidation, and lipid caloric overload reduces hepatic NAD+ levels and triggers lipotoxicity leading to non-alcoholic fatty liver disease (NAFLD). Hepatic NAD+ levels decline with age in humans and rodents, which may contribute to NAFLD susceptibility during aging. Stressing the significance of adequate hepatic NAD+ homeostasis, aberrant NAD+ metabolism is also implicated in alcoholic hepatic steatosis (Dall et all 2022 J Physiol 600.5 pp 1135-1154).

In a further embodiment, the increase in NMN and/or NAD+ provided by the synbiotic combinations/compositions of the disclosure may be used to treat or ameliorate non-alcoholic fatty liver disease (NAFLD), alcoholic hepatic steatosis.

A recent study by Jia et al 2021 Front Physiol. 12:649547, showed that increasing NMN via biochemical administration attenuates renal interstitial fibrosis after acute kidney injury by suppressing tubular DNA damage and senescence. Yet, as noted above, synbiotic administration may provide advantages in safety, nutrition, temporal stability, personalized effects, or synergistic effects not observed in treatments via biochemical administration or by probiotic or prebiotic treatments alone.

### Cardiovascular Health

As described in beginning of the detailed description NAD+ plays a role as a signaling molecule, where an increase in intracellular levels of NAD+ directs cells to make adjustments to ensure survival, including increasing energy production and utilization, boosting cellular repair, and coordinating circadian rhythms.

NAD+ is known to protect against cardiovascular diseases such as, metabolic syndrome, heart failure, ischemia-reperfusion (IR) injury, arrhythmia and hypertension (Lin et al 2021 Clinica Chimica Acta 515 (2021) 104-110). Therefore, in a further aspect of the invention, the increase in NMN and/or NAD+ provided by the combinations/compositions of the invention is effective in the treatment of cardiovascular disease or improving cardiovascular health.

Several preclinical studies have observed that raising NAD+ concentrations in endothelial cells reverses vascular and endothelial dysfunction (Picciotto et al 2016 Aging Cell 15 (3): 522-530). Further studies have observed a beneficial role for NAD+ in cardiovascular function in mouse models, including models of dyslipidemia, ischemia-reperfusion injury, and diastolic heart failure. This link is supported in humans by epidemiological data demonstrating a correlation between dietary intake of the NAD+ precursor niacin (nicotinic acid) and vascular health, including brachial-artery flow-mediated dilation and serum low-density lipoprotein (LDL) concentrations (Kaplon et al 2014 J Appl Physiol 116 (2): 156-163). Nevertheless, synbiotic administration to increase gut and/or systemic NAD+ and/or NMN may provide advantages in safety, nutrition, temporal stability, personalized effects, or synergistic effects not observed in treatments via biochemical administration or by probiotic or prebiotic treatments alone.

Accordingly, in another aspect of the present disclosure, the synbiotic combination and/or composition or methods disclosed herein may be advantageously used in the treatment or prevention of cardiovascular disease or for improving cardiovascular health when compared to a non-administered subject.

### Infections

Reduction in NAD+ metabolites have been observed during infections and may play a role in the modulation of host's immune response (Growth et al 2021 Frontiers in Molecular Biosciences Vol 8 Article 686412).

NAD+ is useful for in the treatment of bacterial infections. This can both be in terms of recovery after an antibiotic treatment where an increase in NMN and/or NAD+ levels facilitates microbiota recovery. Increased NMN and/or NAD+ levels may, however, also increase the uptake of the antibiotics by increasing the proton motive force across cell walls of fast-growing cells (Baquero et al 2022, Frontiers in Mol. Biosci. vol 9 article 861603). In this respect, the synbiotic composition of the present disclosure may be administered in connection with one or more antibiotics to increase the effect of the antibiotic. In one or more embodiments, the synbiotic combination is administered prior to the administration of the antibiotic(s) or together with the antibiotic(s). In certain embodiments, the synbiotic composition is administered post antibiotic treatment to aid the recovery of the microbiota.

In another aspect of the present disclosure, the synbiotic combination and/or composition may be used in the treatment of bacterial infections in a subject in need thereof or at risk thereof.

In one embodiment, the combination and/or composition of the present disclosure is administered in connection with one or more antibiotics. Specifically, in some embodiments, the composition or combination of the present disclosure may be administered prior to the administration of the antibiotic(s) or together with the antibiotic(s).

The term “antibiotic” refers to an antimicrobial substance active against bacteria by killing them or inhibiting their growth. Examples of antibiotics in are penicillin, cephalosporins, macrolides, fluoroquinolones, sulfonamides, stilbenoids, tetracyclines and aminoglycosides and derivatives thereof.

Low NAD+ concentrations associated with risk factors for poor COVID-19 outcomes, but certain viral infections can further deplete NAD+ in infected cells. For example, lower NAD+ concentrations have been reported in human peripheral blood leukocytes infected with HIV-1 in vitro, human fibroblasts infected with herpes simplex virus 1 (HSV-1), or in SARS-COV-2.

In a further embodiment, the increase in NMN and/or NAD+ provided by the combinations/compositions of the present disclosure has antiviral effects. Specifically, the antiviral effect can prevent severe Covid-19 and other infections.

In addition to treatment of bacterial infections as such, NAD+ treatment via biochemical administration has also been shown to be effectful in the treatment of sepsis. Sepsis is a complex disorder caused pathogen-induced hyperinflammation and subsequent immunosuppression as well as endothelial damage are the dominant features responsible for the high modality of sepsis (Ye et al 2022, Nature Nanotechnology 6 Jun. 2022 https://doi.org/10.1038/s41565-022-01137-w).

As noted with respect to other health benefits, in another aspect of the present disclosure, the combination and/or composition and/or methods disclosed herein may be advantageously used in the treatment of sepsis in a subject in need thereof or at risk thereof.

### Neurological

An increase in NAD+ can also be useful for treating or preventing a neurological or neurodegenerative disease in a subject in need thereof or at risk thereof. The neurological disease can for example be selected from the group consisting of Alzheimer's, ischemic brain damage, Parkinson's disease and Huntington's disease (described in WO 2010/039207 and Baquero et al 2022, Frontiers in Mol. Biosci. vol 9 article 861603).

In another aspect of the present disclosure, the synbiotic combination and/or composition comprising a Bifidobacterium sp. capable of growing on a sialylated HMO and having a functional de novo nicotinamide adenine dinucleotide (nad) pathway and at least one fucosylated oligosaccharide (e.g., neutral fucosylated HMO) may be used in the treatment or prevention a neurological or neurodegenerative disease in a subject in need thereof or at risk thereof.

However, synbiotic administration to increase gut and/or systemic NAD+ and/or NMN may provide advantages related to nutrition, temporal stability, personalized effects, or synergistic effects not observed in treatments via oral ingestion of NAD+/NMN or by probiotic or prebiotic treatments alone. For example, in one embodiment, the neurological disease is connected with axonal degradation. The neurological disease can be selected from the group consisting of Alzheimer's, ischemic brain damage, amyotrophic lateral sclerosis (ALS) and Parkinson's disease, Huntington's disease.

### Inflammation

Recent studies showed that inflammation increases CD38-mediated NAD+ degradation activity, which decreases NAD+. Hence, an increase in NAD+ levels via biochemical administration may be effective in reducing or alleviating inflammation (US2022/0062458 and WO 2022/026612). Reduction of inflammation may in particular be effective if associated with CD-38 increases in an individual.

Inflammation may also be caused by viral infections. Many viruses engage the innate immune system to launch an interferon response that attacks the NAD+ system, and thereby reduce NAD+ availability. It has been shown that the NAD precursor NR improves survival of zika-infected mice by increasing NAD+.

In another aspect of the present disclosure, the synbiotic combination and/or composition of the present invention may be advantageously used in reduction of inflammation or symptoms thereof in a subject in need thereof when compared to a non-administered subject.

### Muscle Repair or Regeneration

Muscle degeneration or muscle atrophy is the decline of the skeletal muscle mass which can be partial or complete. It can be caused by a number of aspects which includes aging, lack of physical activity, malnutrition, and genetics. Muscle atrophy or degeneration is common in athletes. It occurs as a consequence of over-use, trauma, immobilization or lack of use after sports injuries where the strength of the muscle is lost. In general, muscles have adequate repair capacity particularly in young people, but this repair process can become ineffective after repeated rounds of over-use, severe trauma or other processes. In such cases the muscles lose function and strength of contraction and can be replaced by scar tissue The scar tissue lacks contractility and causes loss of muscle function. Increasing NAD+ via biochemical administration in damaged or stressed muscle cells has a positive effect on their regeneration (see for example WO 2022/026612). Yet, synbiotic administration to increase gut and/or systemic NAD+ and/or NMN may provide advantages in safety, nutrition, temporal stability, personalized effects, or synergistic effects not observed in treatments via biochemical administration or by probiotic or prebiotic treatments alone.

Accordingly, in another aspect of the present disclosure, the synbiotic combination and/or composition may be beneficially used for improving muscle repair or regeneration, in particular in a subject with a physical injury or accident, muscle immobilization, muscle overuse, loss of blood circulation, or lack of muscle use after injury, when compared to a non-administered subject.

The combination and/or composition of the present disclosure may be particularly useful for athletes with a high physical activity level.

The central component of physical fatigue which is not associated with a specific disease appears to be triggered by an increased serotonin level in the central nervous system. During motor activity, serotonin released in the synapses that contact motoneurons to promote muscle contraction. When the level of motor activity is high, the amount of serotonin released increases and spill over occurs. The serotonin then binds to extra synaptic receptors located on the axon initial segment of motoneurons with the result that nerve impulse initiation and thereby muscle contraction are inhibited. Since NAD+ is known to increase serotonin, it may be used to prevent or relieve physical fatigue. Furthermore, during exercise NADH and oxidative stress increase, increasing NMN and/or NAD+ via biochemical administration can improve the antioxidation in a subject when compared to a non-administered subject, in particular during or after exercise. However, synbiotic administration to increase gut and/or systemic NAD+ and/or NMN may provide advantages in safety, nutrition, temporal stability, personalized effects, or synergistic effects not observed in treatments via biochemical administration or by probiotic or prebiotic treatments alone.

Thus, in another aspect of the present disclosure, the synbiotic combination and/or composition disclosed herein may be advantageously used to improve antioxidation in a subject and/or relieving physical fatigue.

### Skin and Hair Health

In US2022/0062458 it has been shown that yeast powder rich in NMN improves skin condition, for example by enhancing skin firmness, reducing wrinkles and reducing skin roughness and hair health for example by reducing hair loss or hair scantiness. Furthermore, benefits in antiinflammation, antioxidation, and/or anti-aging, was described.

In another aspect of the present invention the combination and/or composition of the present invention is accordingly used for improving adverse skin condition and improving hair health of a subject when compared to a non-administered subject. Specifically, skin conditions to be improved encompass enhancing skin firmness, reducing skin aging, reducing wrinkles, improving the signs of photoaging, reducing hyperpigmentation, reducing itchy skin and reducing skin roughness. With respect to hair health, for example, reducing hair loss or hair scantiness may be improved.

### DNA Damage Repair

Cellular NAD+ is required for base excision repair to maintain genome stability and to mount a robust cellular response to DNA damage. A cellular or organismal reduction in NAD+ is not only associated with aging and age-related diseases but is a prevalent phenotype in cancer as well, suggesting that cancer-related DNA repair defects may be, in part, the result of NAD+biosynthesis deficit (Jia et al 2021 Front Physiol. 12:649547).

In another aspect of the present invention the increase in NMN and/or NAD+ provided by the combination and/or composition of the present invention is used to improve base excision repair, genome stability maintenance, DNA damage repair, and cancer prevention.

It is well known that NAD+ increase the growth of many cancers, however in the present disclosure increased NMN and/or NAD+ via administration of the disclosed synbiotic combinations may be advantageously used in a preventive manner to prevent DNA damage to cells that may be the cause of some age-related cancers.

### Short Chain Fatty Acid (SCFA)

In another aspect of the present disclosure, the synbiotic combination and/or composition may be advantageously used to increase the abundance of the SCFA's acetate, propionate and/or butyrate in the gut of the subject, when compared to a non-administered subject. In various embodiments, the subject is a youth, adult, or an older adult. In certain embodiments, the subject is an infant or a toddler.

In certain embodiments a “subject” may be a human or a mammal, or other domestic animals such as pets (cats, dogs, rodents, rabbits, avian species, reptiles, etc.), livestock and performance animals (pigs, poultry, goat, sheep and cows) and working animals (horses, oxen, camels, donkeys and elephants) with a gut microbiome. Preferably, the subject is a human, such as a non-infant human. The term “non-infant human” or “non-infant” means a human of 3 years of age and older. A non-infant human can be a child, a teenager, an adult or an elderly. The term “elderly” in the context of a human means an age from birth of at least 60 years, preferably above 65 years, more preferably above 70 years. The term “older adult” in the context of a human means an age from birth of at least 40 years, preferably above 50 years, more preferably above 55 years, and includes elderly individuals.

The terms “treat” or “treatment” or “treating” as used herein refers to both treatment of an existing disease (e.g., a disease, condition or disorder as herein referred to) or prevention of a disease, i.e. prophylaxis. It will therefore be recognized that treatment as referred to herein may, in some embodiments, be prophylactic and in addition includes ameliorating, mitigating, slowing, arresting, preventing or reversing a disease, condition or disorder. Maintenance and/or promotion of health in an individual not suffering from a disease but who may be susceptible to the development of an unhealthy condition is also considered treatment in the context of the present invention. Specifically, the treatment addresses a condition, disorder or disease with the objective of improving or stabilizing an outcome in the person being treated. Treatment includes the dietary or nutritional management of the medical condition or disease by addressing nutritional needs of the person being treated.

An “effective amount” of a synbiotic composition of the present invention means an amount of a composition of at least one Bifidobacterium sp. capable of growing on a sialylated HMO and having a functional de novo nicotinamide adenine dinucleotide (nad) pathway and at least one fucosylated oligosaccharide that render a desired treatment outcome in the subject it is administered to. An effective amount can be administered in one or more doses to achieve the desired treatment outcome.

An “effective amount” of a Bifidobacterium sp. capable of growing on a sialylated HMO and having a functional de novo nicotinamide adenine dinucleotide (nad) pathway means an amount that when feed on effective amount on fucosylated oligosaccharide, is sufficient to secrete NAD+ and/or NMN in the gut of the subject in an amount that is sufficient to render a desired treatment outcome in the subject it is administered to.

An “effective amount” of a fucosylated oligosaccharide means an amount that provides an fucosylated oligosaccharide, such as a fucosylated HMO, in a sufficient amount to positively affect the NAD+ secretion of the probiotic with which it is administered.

“Enteral administration” means any conventional form for delivery of a composition to a subject that causes the deposition of the composition in the gastrointestinal tract (including the stomach). Methods of enteral administration include feeding through a naso-gastric tube or jejunum tube, oral, direct delivery to the gut, sublingual and rectal.

“Oral administration” means any conventional form for the delivery of a composition to a non-infant through the mouth. Accordingly, oral administration is a form of enteral administration.

“Topical administration” means any conventional form for the delivery of a composition to the skin, such as in particular in the form of a dermatological or cosmetic composition. Such compositions generally comprise the synbiotic composition according to the present invention and ad a suitable cosmetically acceptable carrier.

The proper dosage of the synbiotic composition/combination of the present disclosure may be determined, at least in part, based upon factors such immune status, body weight and age. In some cases, the dosage of the fucosylated oligosaccharide, such as the fucosylated HMO will be similar to that found for the specific HMO in human breast milk. The required amount of fucosylated oligosaccharide would generally be in the range from about 1 g to about 20 g per day, in certain embodiments from about 2 g to about 15 g per day, from about 3 g to about 10 g per day, in certain embodiments from about 1 g to about 10 g per day. Appropriate dose regimes can be determined based on the present disclosure and/or on factors known to a person of ordinary skill in the art. The dosage of the probiotic within the composition/combination of the present disclosure is expressed as cfu/day. In the context of the present disclosure, the amount of the probiotic is expressed as colony forming units (cfu), which means the number of viable cells (i.e., probiotic cells which are able to multiply via binary fission under the controlled conditions). In one embodiment, the probiotic dose can be higher than 1E+08 and lower than 1E+12 cfu/day. In an embodiment, the probiotic dose can be of at least 1E+08, 2E+08, 3E+08, 4E+08, 5E+08, 6E+08, 7E+08, 8E+08, 9E+08, 1E+09, 2E+09, 3E+09, 4E+09, 5E+09, 6E+09, 7E+09, 8E+09, 9E+09, 1E+10, 2E+10, 3E+10, 4E+10, 5E+10, 6E+10, 7E+10, 8E+10, 9E+10, 1E+11, 2E+11, 3E+11, 4E+11, 5E+11, 6E+11, 7E+11, 8 E+11, 9E+11, or 1E+12 cfu/day. In another embodiment, the probiotic dose can be of no more than 1E+12, 9E+11, 8E+11, 7E+11, 6E+11, 5E+11, 4E+11, 3E+11, 2E+11, 1E+11, 9E+10, 8E+10, 7E+10, 6E+10, 5E+10, 4E+10, 3E+10, 2E+10, 1E+10, 9E+09, 8E+09, 7E+09, 6E+09, 5E+09, 4E+09, 3E+09, 2E+09, 1E+09, 9E+08, 8E+08, 7E+08, 6E+08, 5E+08, 4E+08, 3E+08, 2E+08, or 1E+08 cfu/day. In yet another embodiment, the probiotic dose can be of between 1E+08, 2E+08, 3E+08, 4E+08, 5E+08, 6E+08, 7E+08, 8E+08, 9E+08, 1E+09, 2E+09, 3E+09, 4E+09, 5E+09, 6E+09, 7E+09, 8E+09, 9E+09, 1E+10, 2E+10, 3E+10, 4E+10, 5E+10, 6E+10, 7E+10, 8E+10, 9E+10, 1E+11, 2E+11, 3E+11, 4E+11, 5E+11, 6E+11, 7E+11, 8 E+11, 9E+11, or 1E+12 or 1E+12, 9E+11, 8E+11, 7E+11, 6E+11, 5E+11, 4E+11, 3E+11, 2E+11, 1E+11, 9E+10, 8E+10, 7E+10, 6E+10, 5E+10, 4E+10, 3E+10, 2E+10, 1E+10, 9E+09, 8E+09, 7E+09, 6E+09, 5E+09, 4E+09, 3E+09, 2E+09, 1E+09, 9E+08, 8E+08, 7E+08, 6E+08, 5E+08, 4E+08, 3E+08, 2E+08, or 1E+08 cfu/day. In a further embodiment, the probiotic dosage is of between 5E+08 and 5E+10 cfu/day. In a yet further embodiment, the probiotic dosage is of between 1E+09 and 5E+10 cfu/day.

Various embodiments of present disclosure are described in the following clauses:
1. A combination comprising at least one Bifidobacterium sp. having a functional de novo nicotinamide adenine dinucleotide (nad) pathway and at least one fucosylated oligosaccharide for use in increasing nicotinamide mononucleotide (NMN) and/or nicotinamide adenine dinucleotide (NAD+) in the gut or the skin of a subject, when compared to a non-administered subject.
2. The combination according to clause 1, wherein the at least one Bifidobacterium sp is capable of growing on a sialylated HMO.
3. The combination according to clause 2, wherein the sialylated HMO is a sialyllactose such as 3′SL or 6′SL.
4. The combination according to clause 1 to 3, wherein the subject is an animal or a human.
5. The combination according to any one of the preceding clauses, wherein the human is a non-infant.
6. The combination according to any one of the preceding clauses, wherein the combination is delivered by enteral or topical administration, preferably oral administration or direct delivery to the gut.
7. The combination according to any one of the preceding clauses, wherein the fucosylated oligosaccharide is selected from the group consisting of 2′FL, 3FL, DFL, FSL LNFP-I, LNFP-II, LNFP-III, LNFP-V, LNFP-VI, LNDFH-I, LNDFH-II and LNDFH-III or a mixture thereof.
8. The combination according to any one of the preceding clauses, wherein the fucosylated oligosaccharide is a fucosyllactose, such as 2′FL, 3FL, DFL or a mixture thereof.
9. The combination according to any one of the preceding clause, wherein the at least one Bifidobacterium sp. comprises the following functional genes nadC, nadD and nadE of the nad pathway.
10. The combination according to clause 9, wherein the functional genes encode for polypeptides having the following enzymatic activities:
   a) carboxylating nicotinate-nucleotide diphosphorylase (nadC) or a functional variant thereof, and
   b) nicotinate-nucleotide adenylyltransferase (nadD) or a functional variant thereof, and
   c) ammonia-dependent NAD(+) synthetase (nadE) or a functional variant thereof.
11. The combination according to any one of the preceding clauses, wherein the at least one Bifidobacterium sp. comprises an intact de novo nad pathway.
12. The combination according to clause 11, wherein the at least one Bifidobacterium sp. comprises the following genes nadB, nadA, nadC, nadD and nadE of the nad pathway.
13. The combination according to any one of clauses 11 or 12, wherein the genes of the intact de novo nad pathway encode for polypeptides having the following enzymatic activities:
   a) L-aspartate oxidase (nadB) or a functional variant thereof, and
   b) quinolinate synthase (nadA) or a functional variant thereof, and
   c) carboxylating nicotinate-nucleotide diphosphorylase (nadC) or a functional variant thereof, and
   d) nicotinate-nucleotide adenylyltransferase (nadD) or a functional variant thereof, and
   e) ammonia-dependent NAD(+) synthetase (nadE) or a functional variant thereof.
14. The combination according to any one of the preceding clauses, wherein the at least one Bifidobacterium sp. can grow on one or more HMOs selected from the following groups: neutral non-fucosylated HMOs, neutral fucosylated HMOs, sialylated HMOs, or a mixture thereof.
15. The combination according to clause 14, wherein the at least one Bifidobacterium sp. can grow on at least one HMO from each group of HMOs.
16. The combination according to clause 14 or 15, wherein the at least one Bifidobacterium sp. can grow on any one of the HMOs selected from the group consisting of LNT-II, LNT, LNnT, 2′FL, 3FL, DFL, LNFP-1, 3′SL and 6′SL.
17. The combination according to clause 14 to 16, wherein the at least one Bifidobacterium sp. can grow on all of the following HMOs LNT, LNnT, 2′FL, 3FL, DFL, 3′SL and 6′SL.
18. The combination according to any one of the preceding clauses, wherein the at least one Bifidobacterium sp is selected from the group consisting of Bifidobacterium bifidum or Bifidobacterium longum.
19. The combination according to clause 18, wherein the at least one Bifidobacterium longum is the Bifidobacterium longum subspecies infantis.
20. The combination according to clause 18 or 19, wherein the at least one Bifidobacterium bifidum is Bifidobacterium bifidum DSM 32403 or Bifidobacterium bifidum HA-132 (CNCM I-5898).
21. The combination according to any of the preceding clauses, wherein the increase in NMN and/or NAD+ is useful for treating or preventing a mitochondria-related disease or a condition associated with altered mitochondrial function in a subject in need thereof or at risk thereof.
22. The combination according to clause 21, wherein the mitochondria-related disease or condition is selected from the group consisting of deleterious effects of stress, hyperlipidemia, cognitive disorder, stress-induced or stress-related cognitive dysfunction, mood disorder, anxiety disorder, acute kidney injury, chronic kidney disease, kidney failure, trauma, infection, hearing loss, macular degeneration, myopathies and dystrophies, and combinations thereof.
23. The combination according to any of clauses 1 to 20, wherein the increase in NMN and/or NAD+ is effective against obesity, overweight, reduced metabolic rate, insulin resistance, metabolic syndrome, type 2 diabetes mellitus and complications from diabetes of a subject, when compared to a non-administered subject.
24. The combination according to any of clauses 1 to 20, wherein the increase in NMN and/or NAD+ slows down the aging process, extending the lifespan and vitality of a subject, when compared to a non-administered subject.
25. The combination according to any of clauses 1 to 20, wherein the increase in NMN and/or NAD+ is effective for prevention or delaying the onset of age-related conditions and diseases where aging is a causal factor of a subject when compared to a non-administered subject, such as: age-induced cognition impairment, age-related memory disorder, macular degeneration, vision loss associated with retinal degeneration, cardiovascular diseases, atherosclerosis, gastrointestinal diseases, constipation, diabetes, loss of insulin sensitivity, loss of vitality, reduction of muscle mass and endurance, reduction of bone density, declined immune function, susceptibility to infectious diseases.
26. The combination according to any of clauses 1 to 20, wherein the increase in NMN and/or NAD+ is effective in improving insulin sensitivity in the elderly, when compared to a non-administered elderly.
27. The combination according to any of clauses 1 to 20, wherein the increase in NMN and/or NAD+ is effective in treating or preventing constipation.
28. The combination according to any of clauses 1 to 20, wherein the increase in NMN and/or NAD+ is improving age-related diminished ovary reserve and is effective against ovarian aging of a subject, when compared to a non-administered subject.
29. The combination according to any of clauses 1 to 20, wherein the increase in NMN and/or NAD+ treats or ameliorates non-alcoholic fatty liver disease (NAFLD), alcoholic hepatic steatosis of a subject, when compared to a non-administered subject.
30. The combination according to any of clauses 1 to 20, wherein the increase in NMN and/or NAD+ is effective in the treatment of cardiovascular disease or improving cardiovascular health of a subject, when compared to a non-administered subject.
31. The combination according to any of clauses 1 to 20, wherein the increase in NMN and/or NAD+ is useful for treating or preventing a neurological or neurodegenerative disease in a subject in need thereof or at risk thereof.
32. The combination according to clause 31, wherein the neurological disease is selected from the group consisting of Alzheimer's, amyotrophic lateral sclerosis, ischemic brain damage and Parkinson's disease, Huntington's disease.
33. The combination according to any of clauses 1 to 20, wherein the increase in NMN and/or NAD+ is useful in reducing inflammation or symptoms thereof in a subject in need thereof, when compared to a non-administered subject.
34. The combination according to any of clauses 1 to 20, wherein the increase in NMN and/or NAD+ is useful in improving muscle repair or regeneration, in particular in a subject with a physical injury or accident, muscle immobilization, muscle overuse, loss of blood circulation, or lack of muscle use after injury or relieves physical fatigue, when compared to a non-administered subject.
35. The combination according to any of clauses 1 to 20, wherein the increase in NMN and/or NAD+ improves adverse skin conditions, improves hair health, and/or antioxidation, when compared to a non-administered subject.
36. The combination according to any of clauses 1 to 20, wherein the increase in NMN and/or NAD+ leads to base excision repair, genome stability maintenance, DNA damage repair, and cancer prevention.
37. The combination according to any of clauses 1 to 20, wherein the increase in NMN and/or NAD+ is useful for in the treatment of a bacterial infection or sepsis in a subject in need thereof or at risk thereof, when compared to a non-administered subject.
38. The combination according to clause 37, wherein the combination is administered in connection with one or more antibiotics.
39. The combination according to clause 37, wherein the combination is administered prior to the administration of the antibiotic(s) or together with the antibiotic(s).
40. The combination according to any of clauses 1 to 20, wherein the increase in NMN and/or NAD+ leads to antiviral effects.
41. The combination according to clause 40, wherein the antiviral effect prevents severe Covid-19 and other infections.
42. The combination according to any of clauses 1 to 20, wherein the increase in NMN and/or NAD+ increases the abundance of the SCFA's acetate, propionate and/or butyrate in the gut of the subject, when compared to a non-administered subject.
43. The combination according to any of clauses 1 to 42, wherein the subject is an elderly, infant or a toddler.
44. The combination for the use according to any one of claims 1 to 42, wherein the subject is an elderly.
45. The combination for the use according to any one of claims 1 to 23, 29 and 33 to 42, wherein the subject is an infant or a toddler.
46. The combination according to any of clauses 1 to 45, in the preparation of an anti-aging drug or health or skin care product.
47. A composition comprising at least one Bifidobacterium sp. having an intact de novo nicotinamide adenine dinucleotide (nad) pathway and at least one fucosylated oligosaccharide.
48. The composition according to clause 47, wherein the at least one Bifidobacterium sp. can grow on one or more HMOs selected from the group consisting of a neutral core HMO, a fucosylated HMO and a sialylated HMO.
49. The composition according to clause 48, wherein the at least one Bifidobacterium sp. can grow on a fucosylated oligosaccharide and at least one neutral core HMO or sialylated HMO.
50. The composition according to clause 47 to 49, wherein the fucosylated oligosaccharide is selected from the group consisting of 2′FL, 3FL, DFL, FSL, LNFP-I, LNFP-II, LNFP-III, LNFP-V, LNFP-VI, LNDFH-I, LNDFH-II and LNDFH-III or a mixture thereof.
51. The composition according to clause 47 to 50, wherein the fucosylated oligosaccharide is a fucosyllactose, preferably selected from 2′FL, 3FL, DFL or a mixture thereof.
52. The composition according to any of clauses 47 to 51, wherein the at least one Bifidobacterium sp is a Bifidobacterium Bifidum.
53. The composition according to clause 52, wherein the Bifidobacterium Bifidum is Bifidobacterium Bifidum DSM 32403 or Bifidobacterium bifidum HA-132 (CNCM I-5898).
54. A method comprising (i) selecting a non-infant human having one or more symptoms of an adverse health condition associated with NAD+ deficiency and/or NMN deficiency, (ii) selecting an effective amount of a synbiotic composition comprising at least one Bifidobacterium sp. capable of growing on a sialylated HMO and having a functional de novo nicotinamide adenine dinucleotide (nad) pathway and one or more neutral fucosylated human milk oligosaccharide (HMOs), the synbiotic composition effective for increasing NAD+ and/or NMN in the gut of the non-infant human, and (iii) increasing NAD+ and/or NMN in the gut of the non-infant human by administering the effective amount of the selected HMOs, and (iv) reducing the frequency and/or severity of the one or more symptoms by administering the effective amount of the selected synbiotic composition.

### EXAMPLES

### Example 1

In the present example, seven commercially available probiotic strains (i.e., Bifidobacterium bifidum 32403, Lactobacillus reuteri DSM 12246, Lactobacillus plantarum TIFN101, Bifidobacterium bifidum HA-132, Bifidobacterium bifidum R0071, Bifidobacterium infantis HA-116 and Bifidobacterium infantis R0033) were investigated for their ability to grow on various carbon sources and the supernatants were analyzed using metabolomics to identify interesting metabolites produced by the strains.

### Materials and Methods

Initial overnight cultures in de Man, Rogosa, and Sharpe (MRS) medium with glucose (Merck, cat #110661) were prepared in an anaerobic chamber with mixed gas (90% N₂, 5% CO₂, and 5% H₂) at 37° C.

From the overnight cultures strains were subcultured 1:100 or 1:10 (if slow growing) into fresh MRS (with glucose) and grown to mid-log (without shaking) in the anerobic chamber at 37° C. For the carbohydrate utilization analysis, a BioLector (automated culturomics equipment, Biosciences) was used. Cells were inoculated 1:100 into MRS (without glucose, Liofilchem Cat #610144) in a 48 well MTP plate (Biosciences) containing 1% of one of the following carbon sources: glucose, lactose, a HMO mixture (containing 2′FL, 2′FL: DFL (range of 6:1-8:1), 3FL, 3′SL, 6′SL, LNnT and LNT in equal portions), HMO mixture+lactose or sterile DI water (as no sugar control) in a final volume of 2 mL and grown in the BioLector at 37° C. for up to 72 hours, shaking at 400 rpm under anaerobic conditions and continuous nitrogen gas flow. If a strain grew on the HMO mixture the experiment was repeated with the individual HMOs of the mixture as carbon source. Change in biomass, pH, and dissolved oxygen was monitored every 20 minutes by the BioLector. A minimum of two replicates were completed for each carbon source. Samples were transferred to a 96 deep-well plate, spun down at 4000 rpm for 10 min at 4° C., and supernatants removed for analysis or stored at −80° C. until further analysis.

Sample supernatants were analyzed using metabolomics by Metabolon (NC, USA).

### Results

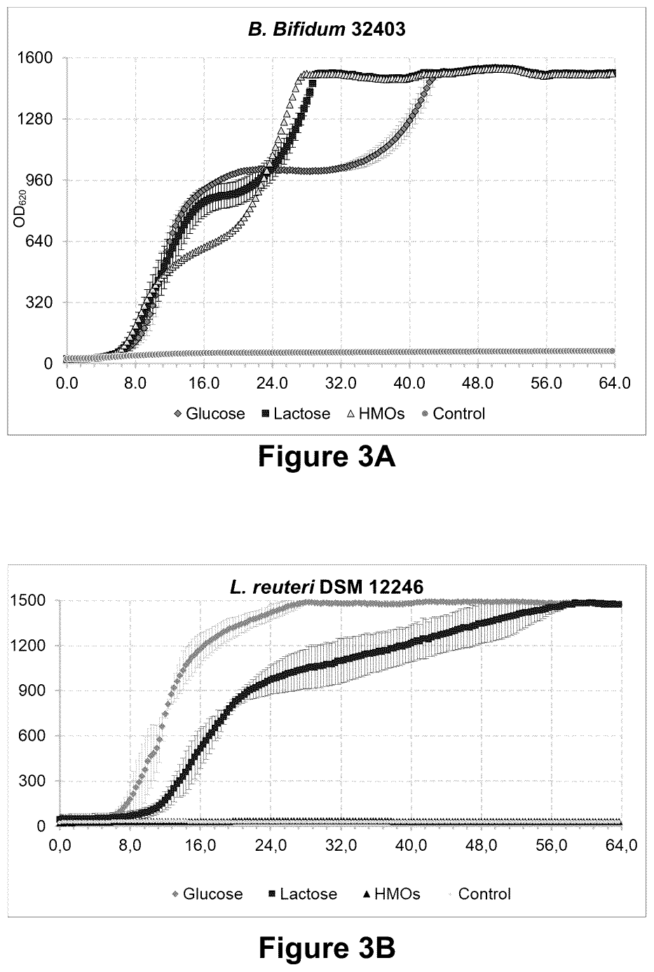

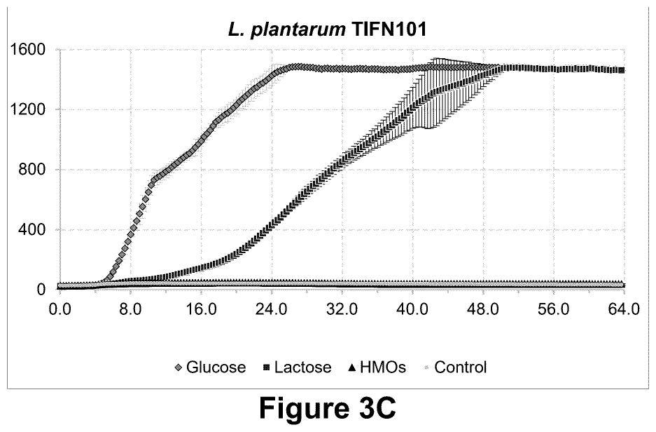

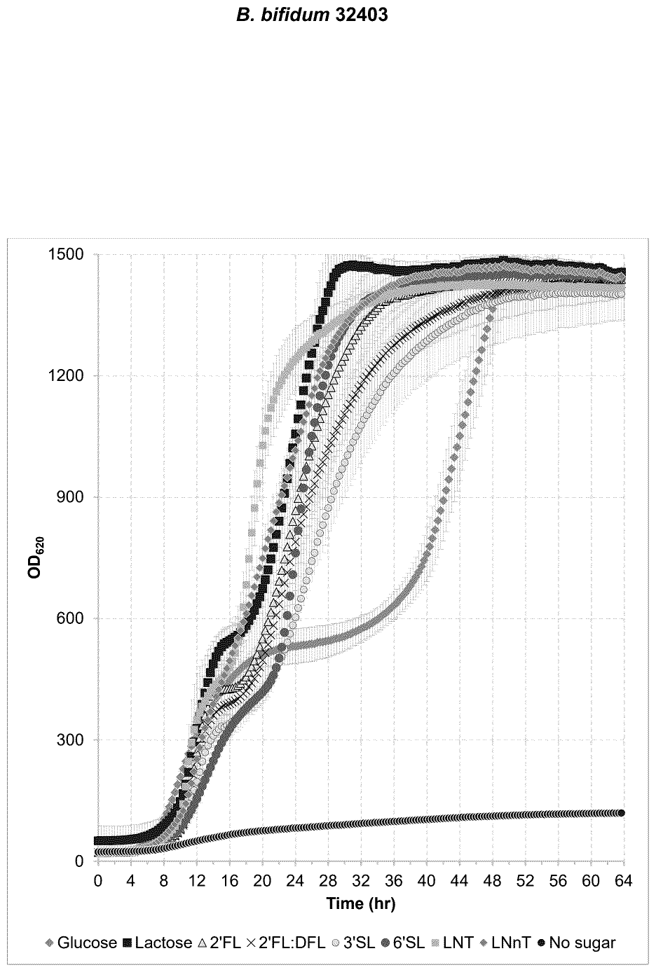

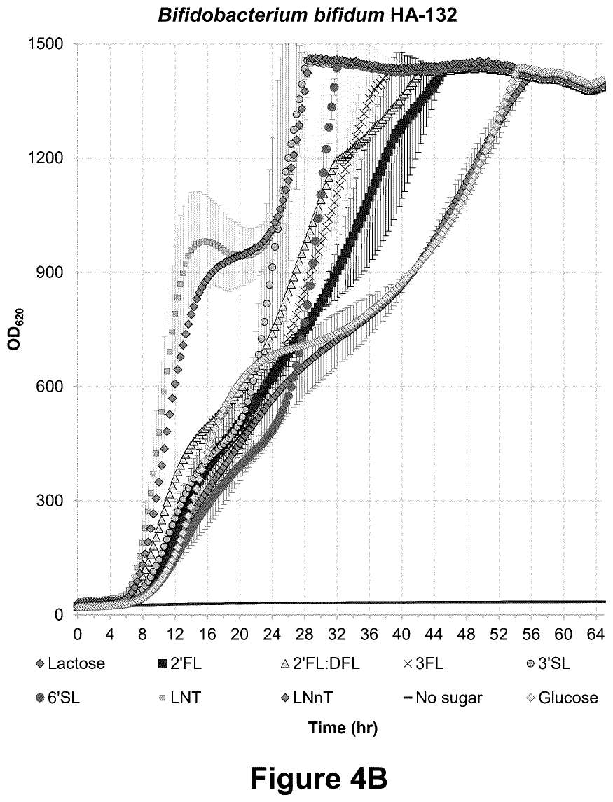

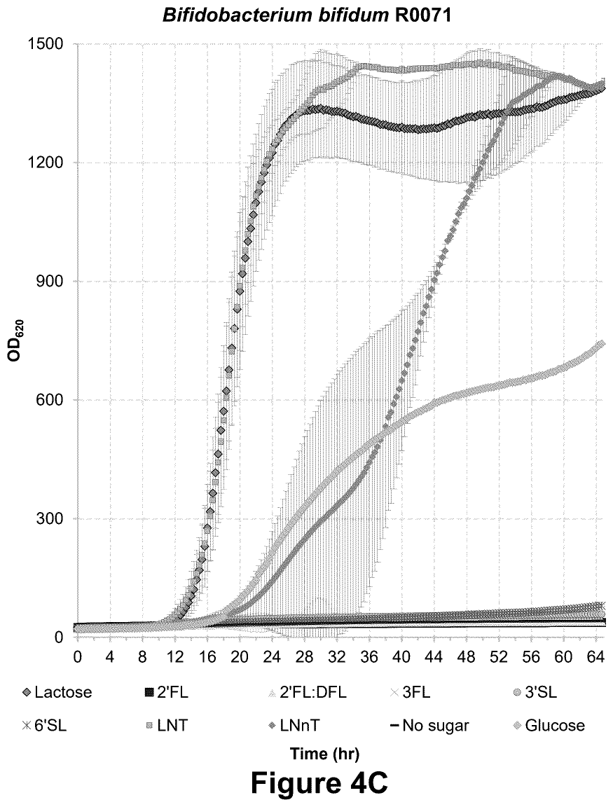

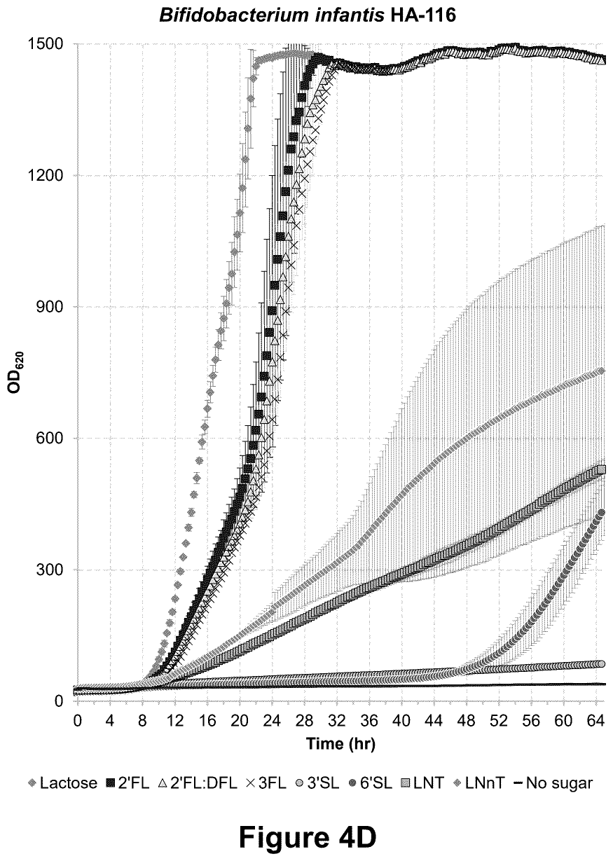

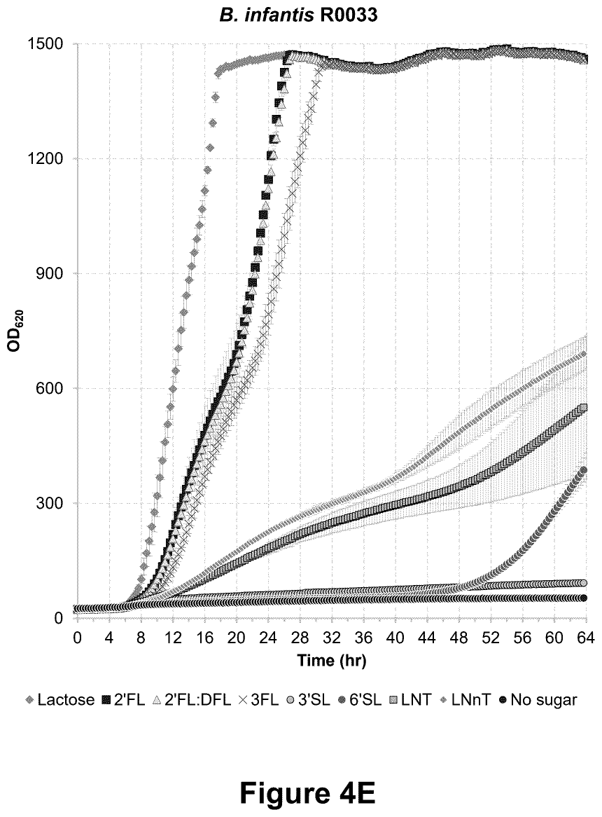

The results from the BioLector growth analysis showed that B. bifidum 32403 was able to grow on the mixture of HMOs (FIG. 3A) whereas L. reuteri DSM 12246 and L. plantarum TIFN101 only were capable of growing in glucose and lactose (FIGS. 3B and 3C respectively) but not on the mixture of HMOs. Since the two lactobacillus strains did not grow on the HMO mixture, their growth was not tested on individual HMOs. FIG. 4 (A-E) shows the growth of five different Bifidobacterium sp. on glucose (A, B and C only), lactose and individual HMOs selected from 2′FL, 2′FL: DFL, 3FL, 3′SL, 6′SL, LNT, LNnT and with no sugar as control. The growth results are summarized in table 1.

TABLE 1
Ability of different Bifidobacterium
sp to grow on different carbon sources.

| Carbon | B. bifidum | B. bifidum | B. bifidum | B. infantis | B. infantis |
|---|---|---|---|---|---|
| source | 32403 | HA-132 | R0071 | HA-116 | R0033 |
| Glucose | ++ | ++ | + | Not tested | Not tested |
| Lactose | +++ | ++ | +++ | +++ | +++ |
| 2′FL | +++ | +++ | − | +++ | +++ |
| 2′FL/DFL | +++ | +++ | − | +++ | +++ |
| 3FL | +++ | +++ | − | +++ | +++ |
| 3′SL | +++ | +++ | − | − | − |
| 6′SL | +++ | +++ | − | − | − |
| LNT | +++ | +++ | +++ | + | − |
| LNnT | +++ | +++ | ++ | + | − |
| No sugar | − | − | − | − | − |

+++ fast exponential growth reaching a plateau before 45 hours
++ slower exponential growth reaching a plateau after 45 hours
+ slow almost linear growth, does not reach plateau during the time of fermentation
− No significant growth for 50 h or more

From this it can be seen that B. bifidum 32403 and B. bifidum HA-132 are capable of growing on neutral core HMOs (i.e., LNT and LNnT), fucosylated HMOs (i.e., 2′FL, 2′FL/DFL and 3FL) and sialylated HMOs (i.e., 3′SL and 6′SL), whereas B. bifidum R0071 only exhibit growth on the neutral core HMOs. The two B. infantis strains both grow well on the fucosylated HMOs and the B. infantis HA-116 also exhibit a slow growth on the neutral core HMOs.

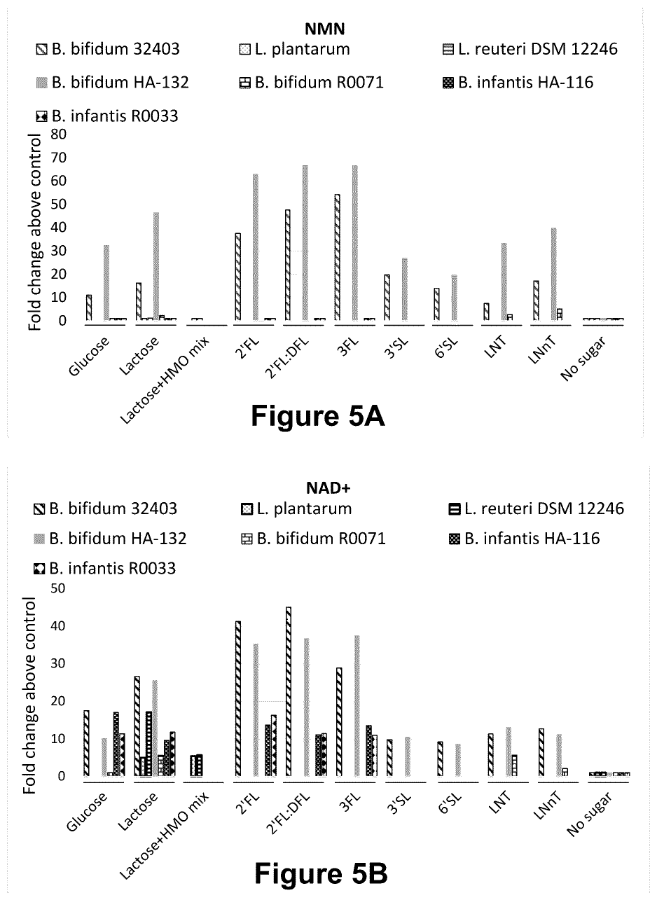

In addition to the difference in carbon source utilization, the metabolomic analysis also revealed that B. bifidum 32403 and B. bifidum HA-132 are capable of producing nicotinamide mononucleotide (NMN) (see FIG. 5A) and nicotinamide adenine dinucleotide (NAD+) (see FIG. 5B) when grown on fucosyllactose (2′FL, 2′FL: DFL or 3FL) which was at least 20 fold above the levels produced when grown on non-fucosylated neutral HMOs (i.e., LNT and LNnT) or on sialylated HMOs (i.e., 3′SL and 6′SL). Excepted for NAD+ produced by B. bifidum 32403 on 3FL, which showed a 1 fold increase, both strains (i.e., B. bifidum 32403 and HA-132) also produced NAD+ and NMN significantly above the production observed when grown on non-fucosylated lactose.

The two lactobacillus strains (i.e., L. reuteri DSM 12246 and L. plantarum TIFN101) however did not produce any NMN independently of the source of carbon with which they were fed. However, these two Lactobacillus strains were able to produce NAD+ when grown on lactose or a combination of lactose+HMO mixture but not on individual HMOs. B. bifidum R0071 was able to produce low amounts of NMN when grown on LNT or LNnT, but below what was observed with the B. bifidum 32403 and B. bifidum HA-132 on glucose and lactose or on the same carbon source. B. bifidum R0071, was also able to produce NAD+ when fed with lactose (but not with the Lactose+HMO mixture) and with neutral core HMOs, but not beyond the levels observed for B. bifidum 32403 and B. bifidum HA-132 when fed with same carbon source.

L. reuteri DSM 12246 produced more NAD+ when grown on lactose only than on the combination of lactose and HMOs indicating that fucosylated HMO did not have any positive effect on NAD+production in this strain. The two B. infantis strains (HA-116 and R0033) did produce a bit more NAD+ when grown on fucosylated HMOs (2′FL, DFL or 3FL), than when grown on lactose, however these levels were still below the NAD+ levels observed for the B. bifidum 32403 and HA-132 when grown on same carbon source.

In conclusion, when B. bifidum 32403 and B. bifidum HA-132 were grown with a fucosyllactose (2′FL, DFL or 3FL) as carbon sources they were capable of producing NAD+ or its precursor NMN at a level exceeding the level produced with lactose as carbon source. This did not happen to the same extent on any other HMO carbon sources, or for any of the tested species.

### Example 2

In the present example, the presence of the genes involved in the different de novo pathways was determined for the following strains: Bifidobacterium bifidum 32403, Bifidobacterium bifidum HA-132, Bifidobacterium bifidum R0071, Bifidobacterium infantis HA-116, Bifidobacterium infantis R0033, Lactobacillus reuteri DSM 12246 and Lactobacillus plantarum TIFN101. Such comparison was determined based on whole genome sequencing data of the seven strains as shown in Table 2.

TABLE 2
Nad+ pathways genes in selected strains and their related NMN and NAD+
effect. NMN and NAD+ effect refers herein to the ability of each strain
to improve its NMN and/or NAD+ production to a level above what is observed
when grown on lactose or glucose using the conditions described in Example 1 (FIG. 5).

| | Bb | Bb | Bb | Bi | Bi | Lr | Lp |
|---|---|---|---|---|---|---|---|
| | 32403 | HA-132 | R0071 | HA-116 | R0033 | 12246 | TIFN101 |
| nadA | + | + | + | + | + | − | − |
| nadB | + | + | + | + | + | − | − |
| nadC | + | + | + | + | + | + | − |
| nadD | + | + | + | + | + | + | + |
| nadE | + | + | + | + | + | + | + |
| NMN effect | High | High | No | No | No | No | No |
| NAD+ effect | High | High | No | No | Moderate | No | No |

A high effect in Table 2 refers to at least 20 fold increase of NAD+ or NMN when grown on 2′FL as compared to growth on lactose or glucose and a moderate effect is between 5 and 20 fold increase of NAD+ or NMN when grown on 2′FL as compared to growth on lactose or glucose.

From this it was observed that all the tested Bifidobacterium sp contain all the genes constituting the de novo nad pathway, namely nadB, nadA, nadC, nadD and nadE, whereas Lactobacillus reuteri DSM 12246 only contain the nadD and nadE genes and Lactobacillus plantarum TIFN101 contain the nadC, nadD and two copies of nadE.

The two lactobacillus strains were not able to grow on a mixture of HMOs (Example 1) and the Bifidobacterium bifidum R0071, Bifidobacterium infantis HA-116 and Bifidobacterium infantis R0033 although having a functional de novo nicotinamide adenine dinucleotide (nad) pathway (i.e., nadC, nadD and nadE) were not capable of growing on 3′SL and 6′SL (table 1).

However, B. bifidum 32403 and B. bifidum HA-132, were capable of growing on sialylated HMOs (Table 1), possess all the genes constituting the de novo nad pathway, and were capable of producing nicotinamide mononucleotide (NMN) and/or nicotinamide adenine dinucleotide (NAD+) in combination with a fucosyllactose (Example 1).

### Example 3

Since the B. bifidum 32403 showed high production of NAD+ and its precursor NMN when grown on a fucosyl containing carbon source it was investigated whether the strain could grow on L-fucose as carbon source and if so whether it would produce NAD+ and its precursor NMN. The growth analysis was conducted in the BioLector as described in Example 1.

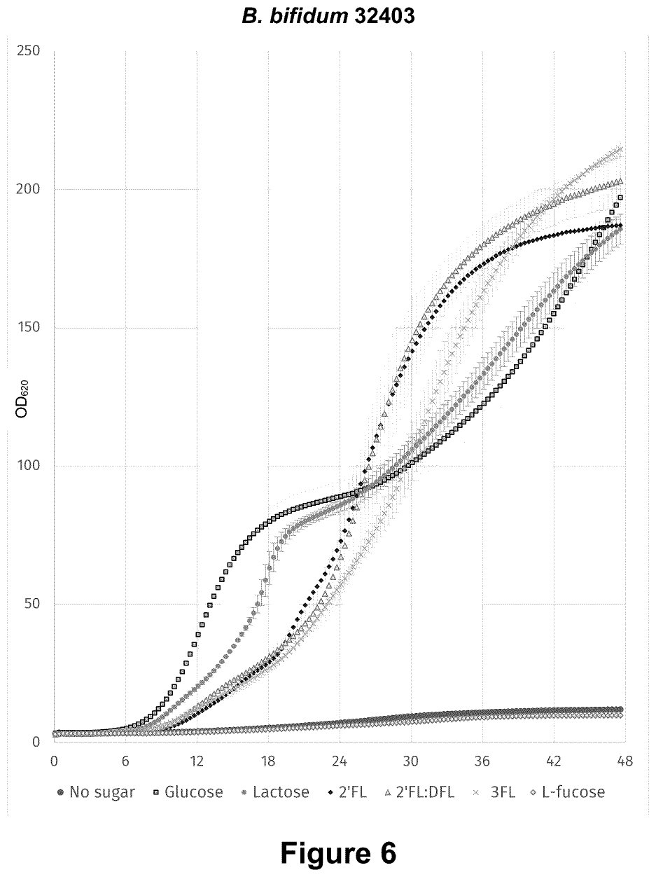

FIG. 6 shows the growth of B. bifidum 32403 on L-fucose or fucosyllactose sources with glucose and no sugar as control. From this it can be seen that the strain is not able to grow on L-fucose alone, indicating that to obtain the increase in NAD+ and its precursor NMN fucosyllactose needs to be present in the growth medium.

### Example 4

Mice lacking the antioxidant transcription factor Nrf2 (Nfe212) develop age-related retinopathy relevant to human age-related macular degeneration (AMD). Here we evaluate the effect of consuming our synbiotic combination on development of features relevant to AMD in Nrf2-null mice. Forty 12-weeks-old male Nrf2-null mice, on a C57BL/6 J genetic background, are individually housed to avoid contamination between mice. Prior to the experiment, the mice are randomly assigned to four groups, ten mice in each group. The mice are fed ad libitum four different experimental diets until 18-months of age.
1. Standard diet (control) (Altromin; no. 1324) (simplified hereon as Nrf2-Ctrl);
2. Synbiotic-containing diet 1 (2′FL: 76 mg/mouse/day; Bifidobacterium bifidum 32403: 10⁸ CFU per gram of feed, Research diet; no. D12492) (simplified hereon as Nrf2-Syn1);
3. Synbiotic-containing diet 2 (2′FL: 76 mg/mouse/day; Bifidobacterium bifidum HA-132: 10⁸ CFU per gram of feed, Research diet; no. D12412) (simplified hereon as Nrf2-Syn2);
4. Synbiotic-containing diet 3 (2′FL: 76 mg/mouse/day; Bifidobacterium infantis R0033: 10⁸ CFU per gram of feed, Research diet; no. D12412) (simplified hereon as Nrf2-Syn3);

Fresh water is administered daily, and all mice have free access to drinking water.

During the course of the study mice were evaluated on a monthly basis for their bodyweight and physical performance by standard tests. Mice that displayed diminished physical performance or drastically reduced bodyweight (loss of >30% of previously measured bodyweight) were removed from the study.

Additionally, fecal and blood samples were collected every 3 months, as well as at the time of euthanasia for assessment of microbiota changes and inflammatory markers, respectively.

Plasma analysis was performed immediately upon sample collection. Fecal samples are frozen and stored at −80° C. until further analysis.

Plasma cytokines (interleukin (IL) 1α, IL1β, tumor necrosis factor (TNF) α, IL6, monocyte chemoattractant protein (MCP)-1, macrophage inflammatory protein (MIP)-1α, IL10, interferon (INF) c, IL15, IL18) are determined in duplicate by using a Bio-Plex Multiplex kit.

To assess the microbiota profile, DNA is extracted from fecal samples and intestinal contents using QIAamp DNA Stool Mini Kit. The DNA concentration of extracts is measured using NanoDrop. The bacterial composition is determined by sequencing. Mice are euthanized by cervical dislocation at 18 months of age.

Mice fed synbiotic diets have less mean concentration in inflammatory markers after throughout the intervention and at the end of the study than mice fed control diet. Also, there is, on average, greater diversity and presence of beneficial microbes of interest in the synbiotic groups than in the control-fed group throughout the intervention and at the time of euthanasia.

In order to assess effects of synbiotic composition on retinal health of Nrf2-null mice, we perform histological analysis of retinas from 18-month-old mice. First, we compare Nrf2-null mice fed Syn1/2/3 or Ctrl diets to WT mice fed identical diets. WT-Syn and -Ctrl or Nrf2-Syn1/2/3 mice have intact, normally laminated retinas and a typical monolayered retinal pigment epithelium (RPE). In comparison, Nrf2-Ctrl mice are present with many retinal abnormalities, including a dramatic thinning of the outer nuclear layer (ONL) and atrophy of the RPE. RPE atrophy is observed both localized or more broadly and is often associated with more dramatically thinned ONL. Some Nrf2-Ctrl retinas develop large lesions that displace the RPE and overlying photoreceptors. We also observe detachment of the RPE from Bruch's membrane, hypopigmentation of the RPE, and dysmorphia of the RPE in Nrf2-Ctrl retinas. Together, the retinal phenotypes in Nrf2-Ctrl mice resemble those seen in dry AMD in humans, particularly RPE atrophy, photoreceptor loss, pigmentary changes, deposits, and RPE dysmorphia.

Another indicator of AMD in humans is pigmentary abnormalities, both hyper and hypopigmentation, which may be related to earlier RPE changes. We find that Nrf2-Ctrl mice have broad areas of hypopigmentation as well as hyperpigmentation, whereas Nrf2-Syn1/2/3 mice have more typical fundus appearances of 18-mo. mice. Histological analysis of the RPE reveals overall quantitative hypopigmentation of Nrf2-Ctrl RPE, as well as RPE thinning and atrophy. We observe a linear relationship between RPE hypopigmentation or RPE thinning and retina damage score, consistent with the relationship between RPE and photoreceptor degeneration that is seen in atrophic AMD in people.

Several additional hallmarks of AMD are visualized in electron micrographs (EM) of 18-mo. RPE. Nrf2-Syn1/2/3 RPE often show several large spindle-shaped pigment granules (melanosomes) and intact mitochondria overlying the basal infoldings, similar to the appearance of WT Syn1/2/3-fed mice and healthy younger mice. In contrast, Nrf2-Ctrl RPE have many disease features usually observed in mouse AMD models, including large basal laminar deposits, accumulation of lipofuscin granules, and loss or displacement of basal infoldings. Incidences of hyperpigmentation and hypopigmentation seen in fundus imaging and histology are also evident at the cellular level. We also observe severe mitochondrial degeneration in Nrf2-Ctrl RPE, as indicated by vacuolated and swollen mitochondria. Finally, we occasionally observe infiltrating cells, likely monocytes/macrophages, between the RPE plasma membrane and Bruch's membrane. Recruitment of macrophages to Bruch's membrane has been observed in human AMD, sometimes in association with lipid-filled debris.

Nrf2-null mice that consumed Ctrl diet develop atrophic AMD, characterized by photoreceptor degeneration, RPE atrophy and pigmentary abnormalities, basal laminar deposits, and loss of the choriocapillaris. In contrast, Nrf2-null-mice that consumed synbiotic diets do not develop retinal disease phenotypes. Consumption of control diet is associated with accumulation of advanced glycation end-products in the RPE and systemically, whereas consumption of the synbiotic diets is associated with increased levels of anti-glycative and anti-oxidative detoxification machinery. Together our data indicate that the synbiotic-containing diets can activate protective pathways to prevent AMD, even in a genetically predisposed animal.

### Example 5

### Effects of a Synbiotic Formulation in Healthy Aging

Study design: randomized, double-blind, parallel, placebo-controlled trial

### Phase: II

Sample size: 120 participants:
1. 40 participants on the claimed synbiotic composition, namely “5 g per day 2′FL+10¹⁰ CFU per day B. bifidum HA-132”,
2. 40 participants on the claimed synbiotic composition, namely “5 g per day 2′FL+10¹⁰ CFU per day B. bifidum 32403, and
3. 40 participants on the placebo (i.e., maltodextrin).

Study length: 18 months

Visits: Baseline, 1 month, 3 months, 6 months, 12 months and 18 months

Biological samples collected: stool, saliva, blood, urine. Potential biopsy on a subgroup of participants (20)

Population (main inclusion criteria):
Female and Male
65 to 80 years old (inclusive)
BMI between 18.5 and 29.9 (inclusive)

### Study Description:

It is hypothesized that the synbiotic formulation, consumed over an 18-month period, increases levels of nicotinamide mononucleotide (NMN) and/or nicotinamide adenine dinucleotide (NAD+). Additionally, it is hypothesized that the increase in NMN and NAD+ levels is associated with an overall maintenance of wellbeing, promotes a healthy aging, and prevents muscle, bone, visual and cognitive degeneration.

### Study Objectives:

### Primary Objective

To measure the increase of levels of nicotinamide mononucleotide (NMN) and/or nicotinamide adenine dinucleotide (NAD+) in brain and blood of elderly participants, measured by:
Change from baseline in brain NAD+ levels as measured by ³¹P MRI
Change from baseline in blood cellular NAD+ concentration in serum

Hypothesis: The study participants who consume the synbiotic formulation have greater mean increase change in NAD+ levels from baseline to 18 months of intervention than the study participants on the placebo.

### Secondary Objective

Changes in Inflammation and Oxidative Markers from Blood Samples
Including TNF-α, IFN-γ, IL-1B, IL-2, IL-4, IL-6, IL-8, IL-10, Malondialdehyde (MDA), Glutathione (GSH), 8-Hydroxydeoxyguanosine (8-OHdG), Tryptophan, Kynurenine, Total antioxidant capacity, C-reactive protein (hsCRP)
Determination of CD38 activity (nicotinamide dinucleotide (NAD+) catabolic enzyme).

Hypothesis: The study participants who consume synbiotic formulation have less mean concentration in inflammatory markers after 18 months of intervention than the study participants on the placebo.

### Prevention of Mitochondria-Related Disease

Comparison of rate/occurrence of diseases diagnosed during intervention (neurodegenerative diseases, etc.)

Hypothesis: There is, on average, less occurrence of mitochondria-related disease diagnosed in the synbiotic group than in the placebo group after 18 months of intervention.

### Metagenomics and Metabolomics

Changes in microbiome profiles at baseline versus 3 months, 6 months, and 12 months (composition, diversity, and metabolites of interest)
   16S rRNA gene sequencing and shotgun metagenomics
   Strain specific qPCR assays
   Targeted and untargeted metabolomics
      Hypothesis: There is, on average, greater diversity and presence of beneficial microbes and metabolites of interest in the synbiotic group than in the placebo group after 18 months of intervention.

### Gastrointestinal Health

Changes in gastrointestinal health and associated quality of life throughout intervention using Gastrointestinal Symptom Rating Scale (GSRS) and Digestive Association Quality of Life (DQLQ), capture of frequency and consistency of bowel movement (Bristol Stool Scale (BSS))

Hypothesis: Participants in the synbiotic group report, on average, better quality of life and less gastrointestinal symptoms (e.g., constipation) than participants in the placebo group, after 18 months of intervention.

### Cognition and Mental Health

Changes in cognitive status throughout intervention assessed by: Questionnaires (e.g., Mini-Mental State Exam (MMSE), Clock Drawing Test (CDT), etc.)
Changes in mental health state including stress, sleep quality and anxiety assessed by: Questionnaires (e.g., Patient Health Questionnaire-9 (PHQ-9), General Anxiety Disorder-7 (GAD-7), Insomnia Severity Index (ISI), State-Trait Anxiety Inventory (STAI), Profile of Mood States (POMS) or Pittsburgh Sleep Quality Index (PSQI))
Biological markers (e.g., cortisol, melatonin and salivary secretory IgA in saliva) Actigraphy (sleep patterns)
Magnetic Resonance Imaging (MRI) to assess changes in brain structure and function throughout intervention
Tests (e.g., CNS Vital Signs (CNSVS) or Electroencephalography (EEG) to capture brain spontaneous activity at rest (with two conditions of eyes open and closed), hedonic function, and go/no-go tasks.

Hypothesis: Participants in the synbiotic group have beneficial mean, a better protection against cognition degeneration and mental health degradation than participants in the placebo group, after 18 months of intervention, when controlling for their baseline values.

### Eye Health

Evolution of eye health during intervention (general vision). Occurrence of age-related vision loss such as macular degeneration (AMD). Assessed by medical examination.

Hypothesis: Participants in the synbiotic group have a better protection and less degradation of the eye health than participants in the placebo group, after 18 months of intervention, when controlling for their baseline values.

### Muscle and Bone Health

Changes in muscle strength and balance assessed by handgrip muscle strength (HGS) via dynamometer and balance testing
Changes in bone density and body composition via Dual X-Ray Absorptiometry (DXA) and Bioelectric Impedance Analysis (BIA)
Changes in concentration of Vitamin D-25 (OH) D3 from serum samples
Measure of physical activity using Physical Activity Scale for the Elderly (PASE)

Hypothesis: Participants in the synbiotic group have, on average, better muscle strength and balance along with a greater bone density than participants in the placebo group, after 18 months of intervention.

### Exercise Endurance

Changes in time-to-exhaustion assessed submaximal treadmill test (VO₂ₘₐₓ)

Hypothesis: Participants in the synbiotic group have, on average, a longer endurance time during submaximal treadmill exercise and a better recuperation than participants in the placebo group, after 18 months of intervention.

### Metabolic Health

Changes in cardiometabolic parameters (e.g., BMI, waist circumference, fasting lipid profile (total cholesterol, HDL-C and LDL-C, triglycerides, non-HDL-C)), apolipoproteins, serum insulin, HbA1c, HOMA-IR)
Changes in lipidomic and polar metabolites profiles (plasma)
Liver function tests (enzymes and proteins related to liver health)

Hypothesis: Participants in the synbiotic group have beneficial mean and a better protection against metabolic degeneration than participants in the placebo group, after 18 months of intervention, when controlling for their baseline values.

### Skin HEALTH

Changes in skin health throughout intervention assessed by: Objective measurements of skin hydration, elasticity, photoaging, skin barrier function (using methods such as Courage&Khazaka Skin Station, VisiaCR imaging system) Subjective assessment via self-evaluation questionnaires

Hypothesis: Participants in the synbiotic group have a better protection against signs of skin aging and a better skin condition (hydration, elasticity), on average, than participants in the placebo group, after 18 months of intervention, when controlling for their baseline values.

### Exploratory Objectives

Immunity: Compare the number of bacterial and viral infections throughout intervention (Hypothesis: Participants in the synbiotic group will have a lower number of self-reported sick days and a shorter duration of cold/flu episode, on average, than participants in the placebo group, after 18 months of intervention)
Mitochondrial function assessment in skeletal muscle using high resolution respirometry
Determination of autophagic (ATG5 protein) and mitophagic (Parkin protein) markers in the serum

### Safety Objectives

To assess the safety and tolerability of a synbiotic formulation in older populations, measured by:
Changes (outside normal variation) in clinical hematology markers (e.g., Complete Blood Count (CBC) including hemoglobin, hematocrit, red blood cell indices, platelet count, leukocyte count and differential elicited after baseline and throughout intervention
Changes (outside normal variation) in clinical chemistry markers (Comprehensive Metabolic Panel (CMP) including glucose, calcium, albumin, protein, sodium, potassium, bicarbonate, chloride, blood urea nitrogen, creatinine, alkaline phosphatase, alanine amino transferase, aspartate amino transferase and bilirubin) elicited after baseline and throughout intervention
Adverse events (AEs) and Serious adverse events (SAEs) until study completion

We do not expect a significant difference for AEs/SAEs or product tolerance between the synbiotic and placebo group.

Participants in the synbiotic group have, on average, less values outside normal variation for markers associated with organ failure/tissue degeneration than participants in the placebo group.

### Example 6

In Example 3 above it was shown that B. bifidum 32403 is not able to grow on L-fucose alone which was taken as an indication that fucosyllactose needs to be present in the growth medium to obtain NAD+/NMN.

In the present example it was investigated if B. bifidum 32403 and B. bifidum HA-132 would produce NAD+/NADH if L-fucose was added as a supplement to a cell growing on a non-fucosylated carbon source, such as glucose.

The growth analysis was conducted in the BioLector as described in Example 1 with two replicates. NAD+/NADH was measured in the sample supernatants using the abcam NAD/NADH Colorimetric Assay Kit (ab65348), using two technical replicates. In brief the assay works by enzymatically converting NAD+ to NADH. After conversion, NADH is reacted with a developer solution which is measured at OD of 420 nm after 30 minutes. Based on controls provided with the assay kit, the user is able to generate a standard curve that enables calculation of total NAD+/NADH in the test samples. The assay is not able to differentiate between NAD+/NADH, therefore these are reported as one value. The data are shown in table 3 below.

TABLE 3
fold increase in NAD+/NADH relative
to the strains grown without carbon source.

| Carbon source | B. bifidum 32403 | B. bifidum HA-132 |
|---|---|---|
| Glucose (Glu) | 8.7 | 8.3 |
| Glu + Fucose | 6.1 | 5.5 |
| 2′FL | 10.2 | 12.2 |

From the results it can be seen that adding fucose to cells growing on glucose actually reduces the amount of NAD+/NADH produced by the cell compared to the same cell just growing on glucose. Furthermore, it is also confirmed that cells growing on 2′FL has a 1.5- to almost 4-fold higher production of NAD+/NADH which confirms what is seen in FIG. 5B. The data are not directly comparable due to the significant lower sensitivity of the assay used to determine 10 NAD+/NADH in the present example compared to the metabolomics approach used in Example 1.

1.-41. (canceled)

42. A composition comprising Bifidobacterium sp. and at least one fucosylated oligosaccharide, wherein the Bifidobacterium sp. is Bifidobacterium bifidum DSM 32403 or Bifidobacterium bifidum HA-132 (CNCM I-5898).

43. The composition of claim 42, wherein the at least one fucosylated oligosaccharide is selected from the group consisting of 2′FL, 3FL, DFL, FSL LNFP-I, LNFP-II, LNFP-III, LNFP-V, LNFP-VI, LNDFH-I, LNDFH-II and LNDFH-III; or a mixture thereof.

44. The composition of claim 42, wherein the at least one fucosylated oligosaccharide comprises a fucosyllactose.

45. The composition of claim 42, further comprising a neutral core HMO.

46. The composition of claim 42, wherein the composition is formulated as a dietary supplement.

47. The composition of claim 42, wherein the composition is formulated as a skin care product.

48. A method of increasing nicotinamide mononucleotide (NMN) and/or nicotinamide adenine dinucleotide (NAD+) in the gut or the skin of a subject, comprising administering an effective amount of the composition of claim 42 to the subject.

49. The method of claim 48, wherein the subject is elderly, an infant or a toddler.

50. The method of claim 48, wherein the subject is obese or overweight.

51. The method of claim 48, wherein the subject has a physical injury associated with muscle immobilization, muscle overuse, loss of blood circulation, or lack of muscle.

52. The method of claim 48, wherein the at least one fucosylated oligosaccharide of the composition comprises a fucosyllactose.

53. The method of claim 52, wherein the fucosyllactose comprises 2′FL, 3FL, DFL, or a mixture thereof.

54. The method of claim 48, wherein the method increases nicotinamide mononucleotide (NMN) and/or nicotinamide adenine dinucleotide (NAD+) in the skin of the subject, and wherein administration improves an adverse skin condition, hair health, and/or antioxidation of the subject.

55. The method of claim 48, wherein the administration is oral or directly to the gut.

56. A method of treating an age-related disorder comprising administering an effective amount of the composition of claim 42 to a subject.

57. The method of claim 56, wherein the age-related disorder is selected from the group consisting of age-induced cognition impairment, age-related memory disorder, constipation, loss of vitality, reduction of muscle mass and endurance, reduction of bone density, declined immune function and age-related diminished ovary reserve.

58. The method of claim 56, wherein the subject is elderly.

59. The method of claim 56, wherein the administration is oral or directly to the gut.

60. The method of claim 56, wherein the at least one fucosylated oligosaccharide of the composition comprises a fucosyllactose.

61. A method of increasing the abundance of the SCFA's acetate, propionate and/or butyrate in the gut of the subject comprising administering an effective amount of the composition of claim 42 to the subject.
